(19) United States
(12) Patent Application Publication
(10) Pub. No.: US 2025/0059147 A1
(43) Pub. Date: Feb. 20, 2025

(54) PROCESSES FOR THE PREPARATION OF PHENYLTETRAHYDROFURAN COMPOUNDS

(71) Applicants: Genentech, Inc., South San Francisco, CA (US); Hoffmann-La Roche Inc., Little Falls, NJ (US)

(72) Inventors: Allen Yu HONG, South San Franciscdo, CA (US); Jacob C. TIMMERMAN, San Mateo, CA (US); Francis GOSSELIN, San Mateo, CA (US); Katarzyna Aleksandra PIECHOWICZ, Redwood City, CA (US); Filip PETRONIJEVIC, Burlingame, CA (US); Kurt PUENTENER, Ueken (CH); Anna-Lena GLASS, Basel (CH); Etienne TRACHSEL, Basel (CH)

(73) Assignees: Genentech, Inc., South San Francisco, CA (US); Hoffmann-La Roche Inc., Little Falls, NJ (US)

(21) Appl. No.: 18/816,550

(22) Filed: Aug. 27, 2024

Related U.S. Application Data
(63) Continuation of application No. PCT/US2023/063316, filed on Feb. 27, 2023.
(60) Provisional application No. 63/268,652, filed on Feb. 28, 2022.

(51) Int. Cl. C07D 307/24; B01J 31/20; B01J 31/22; B01J 31/24; C07D 307/12; C07D 307/14; C07D 307/16

(52) U.S. Cl. CPC C07D 307/24; B01J 31/20; B01J 31/2208; B01J 31/2234; B01J 31/24; C07D 307/12; C07D 307/14; C07D 307/16; B01J 2231/32; B01J 2531/004; B01J 2531/822; B01J 2531/824

(57) ABSTRACT

Provided are phenyltetrahydrofuran compounds that are useful as intermediates in the preparation of pharmaceutical compounds and further provided are processes for the preparation of phenyltetrahydrofuran compounds.

### CROSS-REFERENCE TO RELATED APPLICATIONS

This application is a continuation of International Patent Application No. PCT/US2023/063316, filed on Feb. 27, 2023, which claims priority to U.S. Provisional Application No. 63/268,652 filed Feb. 28, 2022, which is hereby incorporated by reference in its entirety.

### FIELD OF THE DISCLOSURE

The field of the disclosure relates generally to phenyltetrahydrofuran compounds useful as intermediates in the preparation of pharmaceutical compounds and processes for the preparation of phenyltetrahydrofuran compounds.

### BACKGROUND

Useful processes for the preparation of certain substituted 2-phenyltetrahydrofuran intermediate compounds are known from U.S. Pat. No. 10,710,994. Examples of such intermediates include the following compounds:

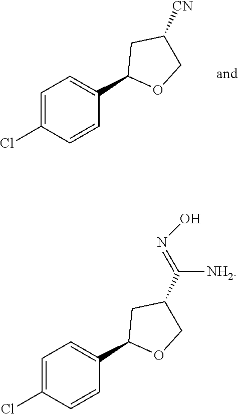 [SMILES: N#C[C@@H]1CO[C@@H](c2ccc(Cl)cc2)C1 ; N/C(=N\O)[C@@H]1CO[C@@H](c2ccc(Cl)cc2)C1]

However, multiple process steps and chromatographic purification of certain of the intermediates of the above compounds are disclosed, and overall yield is relatively low.

A need, therefore, exists for improved processes for preparing substituted phenyltetrahydrofuran compounds for use in preparation of TRPA1 inhibitors as described in U.S. Pat. No. 10,710,994.

### BRIEF DESCRIPTION

In some aspects, the present disclosure is directed to a process for preparing compound (3):

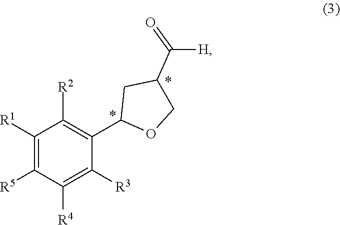

wherein the process comprises step 2: forming a reaction mixture comprising CO, H₂, a rhodium catalyst, a ligand, a solvent, and compound (2), and reacting the reaction mixture to form a reaction product mixture comprising compound (3); wherein compound (2) is of the structure:

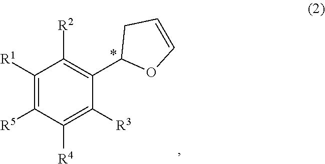

wherein each * independently represents a chiral center; and each of R¹ to R⁵ are independently selected from hydrogen, halo, cyano, unsubstituted C₁-C₆alkyl, substituted C₁-C₆alkyl, unsubstituted C₁-C₆alkoxy, and substituted C₁-C₆alkoxy, wherein at least one of R¹ to R⁵ is halo, wherein at least one halo is selected from Cl and F.

In some other aspects, the present disclosure is directed to a process for preparing compound (4):

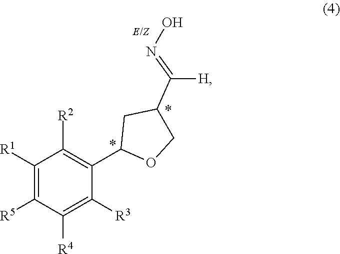

wherein the process comprises step 3: forming a reaction mixture comprising a hydroxylamine solution, a solvent, and compound (3), and reacting the reaction mixture to form a reaction product mixture comprising compound (4); wherein compound (3) is of the structure:

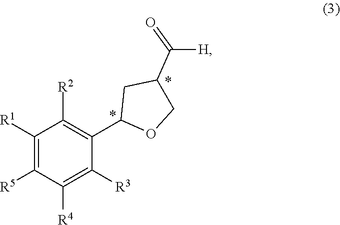

wherein each * independently represents a chiral center; E/Z denotes E/Z isomers; and each of R¹ to R⁵ is independently selected from hydrogen, halo, cyano, unsubstituted C₁-C₆alkyl, substituted C₁-C₆alkyl, unsubstituted C₁-C₆alkoxy, and substituted C₁-C₆alkoxy, wherein at least one of R¹ to R⁵ is halo, wherein at least one halo is selected from Cl and F.

In some other aspects, the present disclosure is directed to a compound of the following structure, or a salt thereof:

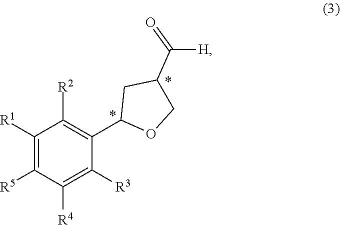

wherein each * independently represents a chiral center; and each of R¹ to R⁵ is independently selected from hydrogen, halo, cyano, unsubstituted C₁-C₆alkyl, substituted C₁-C₆alkyl, unsubstituted C₁-C₆alkoxy, and substituted C₁-C₆alkoxy, wherein at least one of R¹ to R⁵ is halo, wherein at least one halo is selected from Cl and F.

In some other aspects, the present disclosure is directed to a compound of the following structure, or a salt thereof:

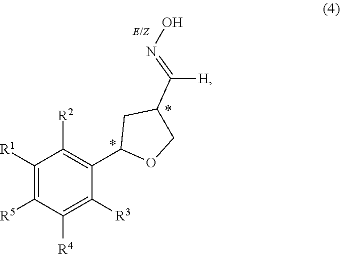

wherein each * independently represents a chiral center; E/Z denotes E/Z isomers; and each of R¹ to R⁵ is independently selected from hydrogen, halo, cyano, unsubstituted C₁-C₆alkyl, substituted C₁-C₆alkyl, unsubstituted C₁-C₆alkoxy, and substituted C₁-C₆alkoxy, wherein at least one of R¹ to R⁵ is halo, wherein at least one halo is selected from Cl and F.

In some other aspects, the present disclosure is directed to a compound of the following structure, or a salt thereof:

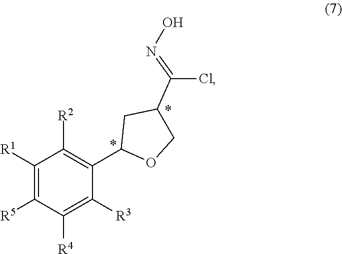

wherein each * independently represents a chiral center; and each of R¹ to R⁵ is independently selected from hydrogen, halo, cyano, unsubstituted C₁-C₆alkyl, substituted C₁-C₆alkyl, unsubstituted C₁-C₆alkoxy, and substituted C₁-C₆alkoxy, wherein at least one of R¹ to R⁵ is halo, wherein at least one halo is selected from Cl and F.

### DETAILED DESCRIPTION

Reference will now be made in detail to certain embodiments of the disclosure, examples of which are illustrated in the accompanying structures and formulas. While the disclosure will be described in conjunction with the enumerated embodiments, it will be understood that they are not intended to limit the disclosure to those embodiments. On the contrary, the disclosure is intended to cover all alternatives, modifications, and equivalents which may be included within the scope of the present disclosure as defined by the claims. One skilled in the art will recognize many methods, processes, and materials similar or equivalent to those described herein, which could be used in the practice of the present disclosure. The present disclosure is in no way limited to the methods, processes, and materials described. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in the art to which this disclosure belongs. Although methods, processes, and materials similar or equivalent to those described herein can be used in the practice or testing of the disclosure, suitable methods, processes, and materials are described below. All publications, patent applications, patents, and other references mentioned herein are incorporated by reference in their entirety.

In some aspects, the present disclosure provides for improved processes for preparing the compounds disclosed herein. As compared to known processes, among other advantages, the present disclosure allows for elimination of chromatographic purification steps which maintaining compound purity and allows for improved yield.

### Definitions

As used herein, the term “alkyl,” by itself or as part of another substituent, means, unless otherwise stated, a straight or branched chain hydrocarbon radical. The number of carbons may suitably be from 1 to 20, from 1 to 12, from 1 to 8, from 1 to 6, or from 1 to 4. Non-limiting examples of alkyl groups include methyl, ethyl, n-propyl, iso-propyl, n-butyl, t-butyl, iso-butyl, sec-butyl, n-pentyl, n-hexyl, n-heptyl, and n-octyl. The term “alkenyl” refers to an unsaturated alkyl radical having one or more double bonds. Similarly, the term “alkynyl” refers to an unsaturated alkyl radical having one or more triple bonds. Non-limiting examples of such unsaturated alkyl groups include linear and branched groups including vinyl, 2-propenyl, crotyl, 2-isopentenyl, 2-(butadienyl), 2,4-pentadienyl, 3-(1,4-pentadienyl), ethynyl, 1—and 3-propynyl, and 3-butynyl, and the higher homologs and isomers.

The terms “alkoxy,” “alkylamino,” and “alkylthio,” are used in their conventional sense, and refer to those alkyl groups attached to the remainder of the molecule via an oxygen atom (“oxy”), an amino group (“amino”) or thio group, and further include mono- and poly-halogenated variants thereof. Additionally, for dialkylamino groups, the alkyl portions can be the same or different.

The terms “cycloalkyl” and “cycloalkylene” refer to a saturated or partially unsaturated carbocyclic moiety having mono- or bicyclic (including bridged bicyclic) rings and 3 to 10 carbon atoms in the ring (i.e., (C₃-C₁₀)cycloalkyl). The cycloalkyl moiety can optionally be substituted with one or more substituents. In particular embodiments cycloalkyl contains from 3 to 8 carbon atoms (i.e., (C₃-C₈)cycloalkyl). In other particular embodiments cycloalkyl contains from 3 to 6 carbon atoms (i.e., (C₃-C₆)cycloalkyl). Non-limiting examples of cycloalkyl moieties include, but are not limited to, cyclopropyl, cyclobutyl, cyclopentyl, cyclohexyl, and cycloheptyl, and partially unsaturated (cycloalkenyl) derivatives thereof (e.g., cyclopentenyl, cyclohexenyl, and cycloheptenyl).

The terms “heterocyclyl” and “heterocycloalkylene” refer to a 4, 5, 6 and 7-membered monocyclic or 7, 8, 9 and 10-membered bicyclic or polycyclic (including bridged bicyclic) heterocyclic moiety that is saturated or partially unsaturated, and has one or more (e.g., 1, 2, 3 or 4) heteroatoms selected from phosphorus, oxygen, nitrogen and sulfur in the ring with the remaining ring atoms being carbon. In some embodiments, the “heterocyclyl” or “heterocycloalkylene” group has 4 to 10 ring atoms, comprising 1, 2, 3 or 4 heteroatoms selected from P, N, O and S, the remaining ring atoms being carbon.

The term “aryl” refers to a cyclic aromatic hydrocarbon moiety having a mono-, bi- or tricyclic aromatic ring of 5 to 16 carbon ring atoms. Bicyclic aryl ring systems include fused bicyclics having two fused five-membered aryl rings (denoted as 5-5), having a five-membered aryl ring and a fused six-membered aryl ring (denoted as 5-6), and having two fused six-membered aryl rings (denoted as 6-6). The aryl group can be optionally substituted as defined herein. Non-limiting examples of aryl moieties include, but are not limited to, phenyl, naphthyl, phenanthryl, indenyl, pentalenyl, and azulenyl. In some embodiments, the aryl group has 6 to 10 carbon ring atoms. In some embodiments, the aryl group has 6 to 12 carbon ring atoms.

The term “heteroaryl” may refer to an aromatic heterocyclic mono- or bicyclic ring system of 5 to 12 ring atoms, comprising 1, 2, 3 or 4 heteroatoms selected from N, O and S, the remaining ring atoms being carbon. Bicyclic heteroaryl ring systems include fused bicyclics having two fused five-membered heteroaryl rings (denoted as 5-5), having a five-membered heteroaryl ring and a fused six-membered heteroaryl ring (denoted as 5-6), and having two fused six-membered heteroaryl rings (denoted as 6-6). The heteroaryl group can be optionally substituted as defined herein. Non-limiting examples of heteroaryl moieties include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, purinyl, pyridopyrimidinyl, pyrrolopyrimidinyl, imidazotriazinyl, pyrazolopyrimidinyl, pyrimidopyridazinyl, pyrimidopyrimidinyl, thiazolopyrimidinyl, pyrazolopyridinyl, imidazopyridazinyl, pyridopyrazinyl, triazolopyrimidinyl, isoxazolopyrimidinyl, and quinoxalinyl.

The substituted and unsubstituted alkyl, alkenyl, alkoxy, alkylamino, and alkylthio moieties may optionally include one or more heteroatoms. As used herein, the term heteroatom is meant to include oxygen (O), nitrogen (N), sulfur(S) and silicon (Si).

Substituents for the alkyl, alkenyl, alkoxy, alkylamino, alkylthio, cycloalkyl, cycloalkylene, heterocyclyl, heterocycloalkylene, aryl, and heteroaryl radicals can be a variety of groups including, but not limited to, -halogen, ═O, —OR′, —NR′R″, —SR′, —SiR′R″R″, —OC(O)R′, —C(O)R′, —CO₂R′, —CONR′R″, —OC(O)NR′R″, —NR″C(O)R′, —NR″′C(O)NR′R″, —NR″C(O)₂R′, —NHC(NH₂)═NH, —NRC(NH₂)═NH, —NHC(NH₂)═NR′, —NR′″C(NR′R″)═N—CN, —NR′″C(NR′R″)—NOR′, —NHC(NH₂)═NR′, —S(O)R′, —S(O)₂R′, —S(O)₂NR′R″, —NR'S(O)₂R″, —NR″′S(O)₂NR′R″, —CN, —NO₂, —(CH₂)₁₋₄—OR′, —(CH₂)₁₋₄—NR′R″, —(CH₂)₁₋₄—SR′, —(CH₂)₁₋₄—SiR′R″R″, —(CH₂)₁₋₄—OC(O)R′, —(CH₂)₁₋₄—C(O)R′, —(CH₂)₁₋₄—CO₂R′, and —(CH₂)₁₋₄CONR′R″, in a number ranging from zero to (2m′+1), wherein m′ is the total number of carbon atoms in such radical. R′, R″ and R′″ each independently refer to groups including, for example, hydrogen, unsubstituted C₁₋₆ alkyl, unsubstituted heteroalkyl, unsubstituted aryl, aryl substituted with 1-3 halogens, unsubstituted C₁₋₆ alkyl, C₁₋₆ alkoxy or C₁₋₆ thioalkoxy groups, or unsubstituted aryl-C₁₄ alkyl groups, unsubstituted heteroaryl, and substituted heteroaryl, among others. When R′ and R″ are attached to the same nitrogen atom, they can be combined with the nitrogen atom to form a 3—, 4-, 5-, 6-, or 7-membered ring. For example, —NR′R″ is meant to include 1-pyrrolidinyl and 4-morpholinyl. Other substituents for alkyl radicals, including heteroalkyl and alkylene, include for example, —O, ═NR′, ═N—OR′, ═N—CN, and ═NH, wherein R′ include substituents as described above. When a substituent for the alkyl radicals (including those groups often referred to as alkylene, alkenyl, alkynyl, heteroalkyl and cycloalkyl) contains an alkylene, alkenylene, or alkynylene linker (e.g., —(CH₂)₁₋₄—NR′R″ for alkylene), the alkylene linker includes halo variants as well. For example, the linker “—(CH₂)₁₋₄—” when used as part of a substituent is meant to include difluoromethylene, 1,2-difluoroethylene, etc.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is meant to include trifluoromethyl, 2,2,2-trifluoroethyl, 4-chlorobutyl, 3-bromopropyl, difluoromethyl, and the like. The term “(halo)alkyl” as used herein includes optionally halogenated alkyl. Thus the term “(halo)alkyl” includes both alkyl and haloalkyl (e.g., monohaloalkyl and polyhaloalkyl). In some embodiments, haloalkyl is C₁-C₆haloalkyl. In some embodiments, haloalkyl is C₁-C₄haloalkyl.

Examples of oxidants within the scope of the present disclosure include, without limitation, N-bromosuccinimide: N-chlorosuccinimide: N-iodosuccinimide: N-chlorosuccinimide: NaOCl: chloramine-T hydrate: 1,3-dichloro-5,5-dimethylhydrantoin: 2-chlorobenzo[d]isothiazole-3(2H) one 1,1-dioxide: CCl₄: CCl₃Br: CB₄: tetraiodomethane: CHI₃: C₂Cl₆: hexachloroacetone: dichloroisocyanuric acid: 1,3,5-trichloro-1,3,5-triazinane-2,4,6-trione; dibromoisocyanuric acid: 1,3,5-tribromo-1,3,5-triazinane-2,4,6-trione: diiodoisocyanuric acid: 2,2,6,6-tetramethylpiperidin-1-yl)oxyl (TEMPO); and 1,3,5-triiodo-1,3,5-triazinane-2,4,6-trione. In some aspects, the oxidant is selected from N-chlorosuccinimide, NaOCl, chloramine-T hydrate, 1,3-dichloro-5,5-dimethylhydrantoin, and 2-chlorobenzo[d]isothiazole-3(2H) one 1,1-dioxide.

As used herein, the term “solvent” refers to any of polar aprotic solvents, polar protic solvents, and non-polar solvents.

As used herein, the term “non-polar solvent” refers to solvents characterized as having a low dielectric constant. Examples include, without limitation, pentane (e.g., n-pentane), hexane (e.g., n-hexane), heptane (e.g., n-heptane), cyclopentane, methyl tert-butyl ether (MTBE), diethyl ether, toluene, benzene, 1,4-dioxane, carbon tetrachloride, chloroform and dichloromethane (DCM). In some aspects, the non-polar solvent has a dielectric constant of less than 2, examples of which include, without limitation, n-pentane, n-hexane and n-heptane. As compared to other non-polar solvents, DCM exhibits some degree of polarity at the bond level (i.e., between carbon and chlorine), but only a small degree of polarity at the molecular level due to symmetry-based cancellation of polarity.

As used herein, the term “polar aprotic solvent” refers to any polar solvent not having a proton-donating ability. Examples include, without any limitation, 2-methyltetrahydrofuran, tetrahydrofuran, ethyl acetate, propyl acetate (e.g., isopropyl acetate, iPrOAc), acetone, dimethylsulfoxide, N,N-dimethylformamide, acetonitrile (CH₃CN), N,N-dimethylacetamide, N-methylpyrrolidone (NMP), hexamethylphosphoramide, and propylene carbonate.

As used herein, the term “polar protic solvent” refers to any polar solvent having a proton-donating ability. Examples include, without limitation: water: C₁₋₅ alcohols such as methanol, ethanol, 1-propanol, 2-propanol, 1-butanol, and 1-pentanol: formic acid: nitromethane; and acetic acid.

As used herein, the term “polar organic solvent” refers to both polar aprotic solvents and polar protic solvents, excluding water.

As used herein, the term “anti-solvent” refers to a solvent in which the referenced compound is poorly soluble and which induces precipitation or crystallization of said compound from solution.

As used herein, the term “organic base” refers to an organic compound containing one or more nitrogen atoms, and which acts as a base. Examples of organic bases include, but are not limited to, tertiary amine bases. Examples of organic bases include, but are not limited to, N-methyl-morpholine (NMM), triethylamine (TEA), N,N′-diisopropylethylamine (DIPEA), and 1,4-diazabicyclo[2.2. 2]octane. In some aspects, the organic base is DIPEA.

As used herein, the term “salts” is meant to include salts of the active compounds which are prepared with relatively nontoxic acids or bases (e.g., those salts that are pharmaceutically acceptable), depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., Pharmaceutical Salts, Journal of Pharmaceutical Science, 1977, 66, 1-19).

Neutral forms of the compounds of the present disclosure can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

Certain compounds of the present disclosure possess asymmetric carbon atoms (optical centers) or double bonds: the racemates, diastereomers, geometric isomers, regioisomers and individual isomers (e.g., separate enantiomers) are all intended to be encompassed within the scope of the present disclosure.

As used herein, the term “chiral” refers to molecules which have the property of non-superimposability of the mirror image partner, while the term “achiral” refers to molecules which are superimposable on their mirror image partner.

As used herein, the term “chiral purity” refers to the mole % of one chiral compound based on the total moles of chiral compounds.

As used herein, the term “stereoisomers” refers to compounds which have identical chemical constitution, but differ with regard to the arrangement of the atoms or groups in space.

As used herein, the term E/Z refers to the IUPAC isomerism convention wherein the substituents at each end of a double bond are assigned priority based on their atomic number. If the high-priority substituents are on the same side of the bond it is assigned Z, and if they are on opposite sides of the bond it is assigned E.

In the structures shown herein, where the stereochemistry of any particular chiral atom is not specified, then all stereoisomers are contemplated and included as the compounds of the disclosure. Where stereochemistry is specified by a solid wedge or dashed line representing a particular configuration, then that stereoisomer is so specified and defined. Unless otherwise specified, if solid wedges or dashed lines are used, relative stereochemistry is intended.

In the description herein, if there is a discrepancy between a depicted structure and a name given to that structure, then the depicted structure controls.

As used herein, the term “reaction mixture” refers to a mixture of reactants. As used herein, the term “reaction product mixture” refers to a mixture of reaction products formed from the reaction mixture.

As used herein, “leaving group” refers to an atom or a group of atoms that is displaced in a chemical reaction as stable species. Suitable leaving groups are well known in the art, e.g., see, March's Advanced Organic Chemistry, 5th Ed., Ed.: Smith, M. B. and March, J., John Wiley & Sons, New York: 2001 and T. W. Greene, Protective Groups in Organic Synthesis, John Wiley & Sons, New York, 1991, the entire contents of each are hereby incorporated by reference. Such leaving groups include, but are not limited to, halogen, alkoxy, sulphonyloxy, optionally substituted alkylsulphonyl, optionally substituted alkenylsulfonyl, optionally substituted arylsulfonyl, and diazonium moieties. Examples of some leaving groups include chloro, iodo, bromo, fluoro, methanesulfonyl (mesyl), tosyl, trifluoromethanesulfonate (i.e., triflate), nitro-phenylsulfonyl (nosyl), and bromo-phenylsulfonyl (brosyl).

As used herein, the terms, “predominantly” and “substantially” refer to greater than 50%, at least 75%, at least 90% at least 95%, or at least 99% on a population %, w/w %, w/v %, v/v %, or mole % basis.

As used herein, unless otherwise indicated, the term “percent yield” refers to yield on a molar basis for the indicated reaction, calculated from actual yield to a theoretical yield based on the reactant that is not in stoichiometric excess. For instance, if 1.0 moles of compound A are reacted with 1.1 molar equivalents of compound B to form 0.9 moles of compound C, the percent yield (based on compound A) would be (0.9)/(1.0)*100=90%.

As used herein, the term “purity,” unless otherwise indicated, refers to the amount of a compound in a sample as compared to the total amount of compounds in the sample. In some aspects, purity may be measured by high pressure liquid chromatography (HPLC) analysis wherein the area % a product represents purity.

As used herein, the terms “area percent” or “area %” in reference to purity refers to the area percent of a peak of a compound in a chromatogram (such as an HPLC chromatogram) as a percentage of the total area of all peaks.

Where the applicant has defined an embodiment or a portion thereof with an open-ended term such as “comprising,” it should be readily understood that (unless otherwise stated) the description should be interpreted to also describe such an embodiment using the terms “consisting essentially of” or “consisting of.”

The transitional phrase “consisting essentially of” is used to define a composition, method or process that includes materials, steps, features, components, or elements, in addition to those literally disclosed, provided that these additional materials, steps, features, components, or elements do not materially affect the basic and novel characteristic(s) of the claims.

As used herein, the terms “comprises,” “comprising,” “includes,” “including,” “has,” “having,” “contains,” “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

When indicating the number of substituents, the terms “at least one” and “one or more” refer to the range from one substituent to the highest possible number of substitution, i.e. replacement of one hydrogen up to replacement of all hydrogens by substituents. The term “substituent” denotes an atom or a group of atoms replacing a hydrogen atom on the parent molecule. The term “substituted” denotes that a specified group bears one or more substituents. Where any group may carry multiple substituents and a variety of possible substituents is provided, the substituents are independently selected and need not to be the same. The term “unsubstituted” means that the specified group bears no substituents. The term “optionally substituted” means that the specified group is unsubstituted or substituted by one or more substituents, independently chosen from the group of possible substituents. When indicating the number of substituents, the terms “at least one” and “one or more” mean from one substituent to the highest possible number of substitution, i.e., replacement of one hydrogen up to replacement of all hydrogens by substituents.

As used herein, the indefinite articles “a” and “an” preceding an element or component of the disclosure are intended to be nonrestrictive regarding the number of instances (i.e., occurrences) of the element or component. Therefore “a” or “an” should be read to include one or at least one, and the singular word form of the element or component also includes the plural unless the number is obviously meant to be singular.

### Preparation of Compound (3)

One aspect of the present disclosure is directed to a process for preparing compound (3) by a hydroformylation reaction. The process comprises step 2: forming a reaction mixture comprising CO, H₂, a rhodium catalyst, a ligand, a solvent, and compound (2), and reacting the reaction mixture to form a reaction product mixture comprising compound (3) according to the following reaction scheme:

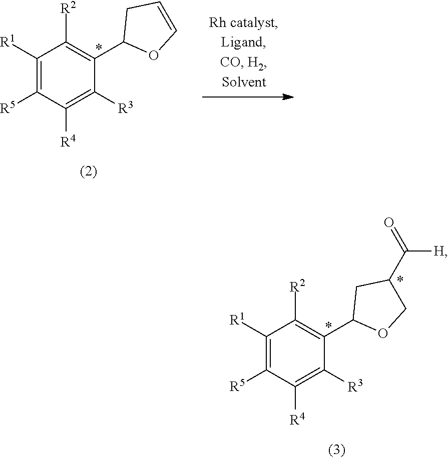

wherein each * independently represents a chiral center and each of R¹ to R⁵ are independently selected from: hydrogen: halo; cyano; unsubstituted and substituted alkyl: unsubstituted and substituted alkenyl: unsubstituted and substituted alkoxy: unsubstituted and substituted alkylamino; and unsubstituted and substituted alkylthio, wherein at least one of R¹ to R⁵ is halo, wherein at least one halo is selected from Cl and F. In some aspects, each of unsubstituted and substituted alkyl, unsubstituted and substituted alkenyl, unsubstituted and substituted alkoxy, unsubstituted and substituted alkylamino, and unsubstituted and substituted alkylthio independently comprise from 1 to 12 carbon atoms, from 1 to 8 carbon atoms, from 1 to 6 carbon atoms, or from 1 to 4 carbon atoms. In some aspects, each of R¹ to R⁵ are independently selected from hydrogen, halo, cyano, unsubstituted C₁-C₆alkyl, substituted C₁-C₆alkyl, unsubstituted C₁-C₆alkoxy, and substituted C₁-C₆alkoxy, wherein at least one of R¹ to R⁵ is halo, wherein at least one halo is selected from Cl and F.

In some aspects wherein at least one of R¹ to R⁵ is halo, at least one halo is Cl. In some aspects wherein at least one of R¹ to R⁵ is halo, at least one halo is F. In some aspects, each of R¹, R², R³, and R⁴ is hydrogen and R⁵ is Cl or F. In some embodiments, each of R¹, R², R³, and R⁴ is hydrogen and R⁵ is Cl. In some embodiments, each of R¹, R², R³, and R⁴ is hydrogen and R⁵ is F.

Rhodium catalysts within the scope of the present disclosure are provided in the form of a compound, such as a hydride, halide, organic acid salt, ketonate, inorganic acid salt, oxide, carbonyl compound, or amine compound, or a combination of two or more thereof. In some aspects, the catalyst is a rhodium carbonyl catalyst. In some aspects, the catalyst is a Rh(I) complex. Non-limiting examples of Rh(I) complexes include Rh(acac)(CO)₂, Rh(acac)(PPh₃)(CO), Rh(acac)((R)-Ph-BPE, Rh(acac)(C₂H₄)₂, Rh(acac)(C₈H₁₄)₂(acetylacetonato (cyclooctene) rhodium (I)), Rh(acac)(COD), bis(1,5-cyclooctadiene) rhodium (I) tetrafluoroborate, bis(1,5-cyclooctadiene) rhodium (I) trifluoromethanesulfonate, bis(norbornadiene) rhodium (I) tetrafluoroborate, chlorobis(cyclooctene) rhodium (I) dimer, Rh₂C₁₂(C₂H₄)₄, Rh₂Cl₂(CO)₄, chloronorbornadiene rhodium (I) dimer, bis(triphenylphosphine) rhodium carbonyl chloride, RhCl(PPh₃)₃, and methoxy (cyclooctadiene) rhodium (I) dimer, or a combination of two or more thereof. As used herein, “acac” is an acetylacetonate group: “OAc” is an acetyl group: “COD” is 1,5-cyclooctadiene; and “Ph” is a phenyl group.

In some aspects of the disclosure, the catalyst is a rhodium carbonyl catalyst. In one aspect, the rhodium catalyst is dicarbonyl (acetylacetonato) rhodium. In another aspect, the rhodium catalyst is Rh(acac)(PPh₃)(CO).

In some embodiments, the mol % ratio of rhodium catalyst to compound (2) is suitably about 0.1 mol %, about 0.25 mol %, about 0.5 mol %, about 0.75 mol %, about 1 mol %, about 1.25 mol %, about 1.5 mol %, about 1.75 mol %, or about 2 mol %, and any range constructed therefrom, such as from about 0.1 mol % to about 2 mol %, from about 0.5 mol % to about 2 mol %, from about 0.5 mol % to about 1.5 mol %, or from about 0.75 mol % to about 1.25 mol %.

In some aspects of the disclosure, rhodium catalyst ligands within the scope of the present disclosure include, for instance and without limitation, mono- and bis-phosphines, mono- and bis-phosphonites, mono- and bis-phosphites, mono- and bis-phosphinites, mono- and bis-phosphoramidites, and mixed phosphoramidite-phosphine ligands. In some aspects, the ligand is selected from mono- and bis-phosphines, mono- and bis-phosphonites, and mono- and bis-phosphites.

One example of a suitable ligand class for the practice of the present disclosure is the BPE family of ligands. Some such ligands are of the structure:

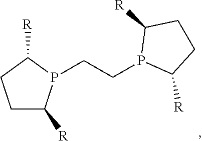

wherein each R is selected from methyl, ethyl, i-propyl, and phenyl. Non-limiting examples of BPE ligands include (R,R)-Me-BPE, (S,S)-Me-BPE, (R,R)-Et-BPE, (S,S)-Et-BPE, (R,R)-Ph-BPE, (S,S)-Ph-BPE, (R,R)-i-Pr-BPE, and (S,S)-i-Pr-BPE. In one aspect, the BPE ligand is (R,R)-Ph-BPE.

Another example of a suitable ligand class for the practice of the present disclosure is the DuPhos family of ligands. Some such ligands are of the structure:

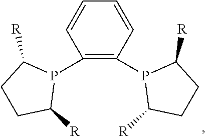

wherein each R is selected from methyl, ethyl, and i-propyl. Non-limiting examples of DuPhos ligands include (R,R)-Me-DuPhos, (S,S)-Me-DuPhos, (R,R)-Et-DuPhos, (S,S)-Et-DuPhos, (R,R)-i-Pr-DuPhos, and (S,S)-i-Pr-DuPhos.

Another example of a suitable ligand class for the practice of the present disclosure is the bisdiazaphos family of ligands. Some such ligands are of the structure:

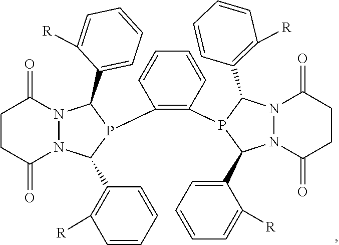

wherein each R is H or bis [(S,S,S)-diazaphos-SPE of the structure:

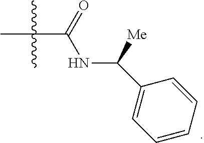 [SMILES: *C(=O)N[C@@H](C)c1ccccc1]

Another example of a suitable ligand class for the practice of the present disclosure is the IndolPhos family of ligands. One non-limiting example of such a ligand is as follows:

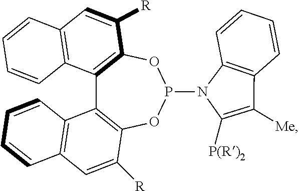

wherein each R is selected from C₁-C₆alkyl, C₃-C₁₀cycloalkyl, C₃-C₁₀cycloalkylene, 4- to 10-membered heterocyclyl having 1, 2, 3 or 4 heteroatoms selected from P, N, O and S, 4- to 10-membered heterocycloalkylene having 1, 2, 3 or 4 heteroatoms selected from P, N, O and S, C₆-C₁₀aryl, and 5- to 10-membered heteroaryl having 1, 2, or 3 heteroatoms selected from O, S, and N, each of which may independently be substituted or unsubstituted. In some aspects, each R is independently selected from methyl, ethyl, i-propyl, and phenyl. In one aspect, each R is methyl. In some aspects, each R′ is independently selected from methyl, ethyl, i-propyl, and phenyl. In one aspect, each R′ is i-propyl. In one aspect, each R is methyl and each R′ is i-propyl.

Another example of a suitable ligand class for the practice of the present disclosure is the ferrocene family of ligands. One non-limiting example of such a ligand is as follows:

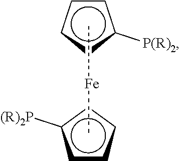

wherein each R is selected from methyl, ethyl, i-propyl, and phenyl: or two R groups are taken together with the P to which they are attached to form a 3-, 4-, 5-, or 6-membered ring which is substituted with two R′, wherein each R′ is independently selected from methyl and ethyl. In some embodiments, each R′ is methyl. In some embodiments, each R′ is ethyl. In one aspect, each R is i-Pr and the ligand is dppf. In some embodiments, two R groups are taken together with the P to which they are attached to form a 4-membered ring which is substituted with two ethyl groups. In some embodiments wherein two R groups are taken together with the P to which they are attached to form a 4-membered ring which is substituted with two ethyl groups, the ligand is (R,R)-Et-FerroTANE. In some embodiments wherein two R groups are taken together with the P to which they are attached to form a 4-membered ring which is substituted with two ethyl groups, the ligand is (S,S)-Et-FerroTANE. In some embodiments, two R groups are taken together with the P to which they are attached to form a 5-membered ring which is substituted with two methyl groups. In some embodiments wherein two R groups are taken together with the P to which they are attached to form a 5-membered ring which is substituted with two methyl groups, the ligand is (R,R)-Me-ferrocelane. In some embodiments wherein two R groups are taken together with the P to which they are attached to form a 5-membered ring which is substituted with two methyl groups, the ligand is (S,S)-Me-ferrocelane. In some embodiments, two R groups are taken together with the P to which they are attached to form a 5-membered ring which is substituted with two ethyl groups. In some embodiments wherein two R groups are taken together with the P to which they are attached to form a 5-membered ring which is substituted with two ethyl groups, the ligand is (R,R)-Et-ferrocelane. In some embodiments wherein two R groups are taken together with the P to which they are attached to form a 5-membered ring which is substituted with two ethyl groups, the ligand is (S,S)-Et-ferrocelane.

Another example of a suitable ligand class for the practice of the present disclosure is the xanthene family of ligands. One non-limiting example of such a ligand is as follows:

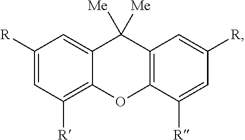

wherein each R is selected from C₁-C₆alkyl, C₃-C₁₀cycloalkyl, C₃-C₁₀cycloalkylene, 4- to 10-membered heterocyclyl having 1, 2, 3 or 4 heteroatoms selected from P, N, O and S, 4- to 10-membered heterocycloalkylene having 1, 2, 3 or 4 heteroatoms selected from P, N, O and S, C₆-C₁₀aryl, and 5- to 10-membered heteroaryl having 1, 2, or 3 heteroatoms selected from O, S, and N, each of which may independently be substituted or unsubstituted and R′ and R″ are independently selected from methyl, ethyl, i-propyl, t-butyl, phenyl, Me₂P—, Et₂P—, i-Pr₂P—, t-Bu₂P—, Ph₂P—, structure (I):

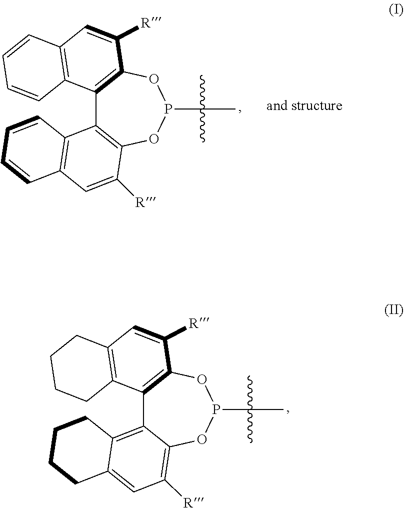

wherein each R′″ is independently selected from hydrogen, C₁-C₆alkyl, C₃-C₁₀cycloalkyl, C₃-C₁₀cycloalkylene, 4- to 10-membered heterocyclyl having 1, 2, 3 or 4 heteroatoms selected from P, N, O and S, 4- to 10-membered heterocycloalkylene having 1, 2, 3 or 4 heteroatoms selected from P, N, O and S, C₆-C₁₀aryl, and 5- to 10-membered heteroaryl having 1, 2, or 3 heteroatoms selected from O, S, and N, each of which may independently be substituted or unsubstituted. In some aspects, each R is independently selected from methyl, ethyl, i-propyl, t-butyl, and phenyl. In one aspect, each R is t-butyl. In one aspect, each R′″ is independently selected from hydrogen, methyl, ethyl, i-propyl, and phenyl. In some embodiments, each R′″ is independently selected from hydrogen, methyl, and phenyl.

In one aspect, each R is t-butyl. In one aspect, R′ is phenyl. In one aspect, R′ is —P(Ph)₂. In one aspect, R″ is of the above structure (I), wherein each R′″ is phenyl. In one aspect, each R is t-butyl, R′ is phenyl, and R″ is of the above structure (I), wherein each R′″ is phenyl. In one aspect, each R is t-butyl, R′ is —P(Ph)₂, and R″ is of the above structure (I), wherein each R′″ is phenyl.

In some embodiments, the equivalent ratio of the ligand to the rhodium catalyst is suitably about 1.1:1, about 1.5:1, about 1.75:1, about 2:1, about 2.25:1, about 2.5:1, about 2.75:1, or about 3:1, and any range constructed therefrom, such as for instance from about 1.1:1 to about 3:1, from about 1.5:1 to about 2.5:1, or from about 1.75:1 to about 2.25:1.

In some aspects, the solvent predominantly comprises at least one non-polar solvent. In some aspects, the solvent predominantly comprises toluene.

In some aspects, the reaction pressure is about 1 bar, about 2 bar, about 3 bar, about 4 bar, about 5 bar, about 6 bar, about 7 bar, about 10 bar, about 15 bar, or about 20 bar, and any range constructed therefrom, such as from about 1 bar to about 20 bar, from about 4 bar to about 15 bar, or from about 5 bar to about 10 bar.

In some embodiments, the reaction temperature is suitably from about 20° C. to reflux. For instance and without limitation, in some embodiments when the solvent predominantly comprises toluene, the reaction temperature is from about 20° C. to reflux, from about 50° C. to reflux, from about 50° C. to about 100° C., or from about 70° C. to about 90° C.

In some aspects, the reaction product mixture comprises compound (3) in solution. In such aspects, the solution of compound (3) may be used directly in a subsequent reaction.

In some aspects, the solution of compound (3) may be worked up such as by, for instance and without limitation, one or more of: filtration; treatment with a metal scavenger: washing with an aqueous phase (e.g., a brine solution); neutralization with an aqueous solution of an acid or base: solvent exchange; phase separation: treatment with an oxidant: crystallization or precipitation by addition of an anti-solvent and optional seed crystal addition and cooling: evaporation: distillation; and drying. In some aspects, compound (3) may be optionally isolated from the reaction product mixture by precipitation and or crystallization to form a slurry, such as by the one or more of the following: addition of an anti-solvent: cooling: pH adjustment; and seed crystal addition. In some embodiments, solid compound (3) may be suitably isolated from the slurry such as by filtration or centrifugation, optionally washed, and optionally dried.

In any of the various aspects of the disclosure, the conversion of compound (2) to compound (3) is at least 80%, at least 85%, at least 90%, or at least 95%.

In any of the various aspects of the disclosure, the purity of compound (3) as measured by high performance liquid chromatography is at least 90 area % or at least 95 area %.

In any of the various aspects of the disclosure, the chiral purity of compound (3) is at least 90 area % or at least 95 area %.

In some aspects of the disclosure, compound (2) is of the structure (2a):

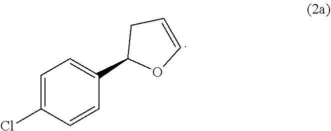 [SMILES: Clc1ccc([C@H]2CC=CO2)cc1]

In some aspects of the disclosure, compound (3) is of the structure (3a):

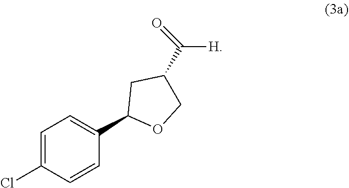

### Preparation of Compound (2)

One aspect of the present disclosure further comprises a process for preparing compound (2) by a Heck arylation reaction. The process comprises step 1: forming a reaction mixture comprising compound (1), 2,3-dihydrofuran, a transition metal catalyst, a ligand, a solvent, a base, and reacting the reaction mixture to form a reaction product mixture comprising compound (2) according to the following reaction scheme:

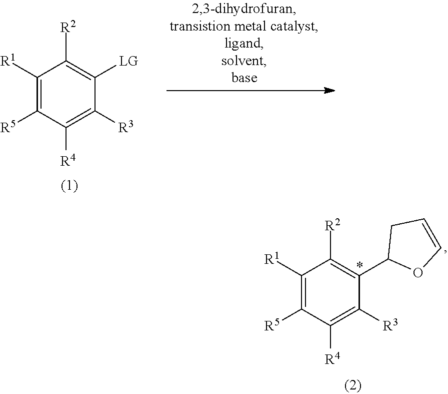

wherein R¹ to R⁵ and the asterisk are as defined elsewhere herein and LG is a leaving group. In some aspects, LG is trifluoromethanesulfonate (triflate).

Transition metal catalysts within the scope of the present disclosure include catalysts such as palladium, platinum, gold, ruthenium, rhodium, and iridium catalysts.

In some aspects, the transition metal catalyst is a palladium catalyst. In some aspects, the palladium catalyst is selected from the group consisting of: [PdCl(X)]₂ wherein X is allyl, cinnamyl or crotyl: [Pd(X) PR′] wherein R′ is alkyl or aryl; [Pd(X)(Y)] wherein X is allyl, cinnamyl or crotyl, Y is cyclopentandienyl or p-cymyl; Pd(dba)₂: Pd₂(dba)₃: Pd(OAc)₂: PdZ₂ wherein Z is Cl, Br or I; Pd₂Z₂(PR)₂ wherein Z is Cl, Br or I, and R′ is alkyl or aryl: Pd(TFA)₂: Pd(dppf) Cl₂; Pd(dppe) Cl₂: Pd₂(dba)₃: Pd(PCy₃)₂Cl₂; Pd(PPh₃)₂Cl₂; Pd(OAc)₂(PPh₃)₂: Pd(PPh₃)₄: Pd(PPh₃)₄C₁₂, Pd(PCy₃)₂: Pd(PCy₃)₂Cl₂; and Pd(t-Bu₃P)₂. In some aspects, the transition metal catalyst is selected from Pd(OAc)₂, Pd(PPh₃)₂Cl₂, and Pd₂(dba)₃. In some such aspects, the transition metal catalyst is Pd(OAc)₂. In some such aspects, the transition metal catalyst is Pd(TFA)₂.

In some embodiments of the disclosure, the mol % ratio of transition metal catalyst to compound (1) is suitably about 0.5 mol %, about 0.75 mol %, about 1 mol %, about 1.25 mol %, about 1.5 mol %, about 1.75 mol %, about 2 mol %, about 2.5 mol %, or about 3 mol %, and any range constructed therefrom, such as from about 0.25 mol % to about 3 mol %, from about 0.75 mol % to about 2.5 mol %, or from about 1 mol % to about 2 mol %.

In some aspects, transition metal catalyst ligands within the scope of the present disclosure include the ligand classes BINAP, WALPHOS, JOSIPHOS, TANIAPHOS, MANDYPHOS, CHENPHOS, MeO-BIPHEP, PPHOS, DUPHOS, TUNEPHOS, SYNPHOS and SEGPHOS. Non-limiting examples of transition metal catalyst ligands include (R)-Segphos, (R)-DM-Segphos, (R)-DTBM-Segphos, P (o-tolyl)₃, P(m-tolyl)₃, (p-tolyl)₃, (R)-2,2′-bis(diphenylphosphino)-1,1′-binaphtyl, (S)-2,2′-bis(diphenylphosphino)-1,1′-binaphtyl, (R)-2,2′-bis(di-p-tolylphosphino)-1,1′-binaphtyl, (S)-2,2′-bis(di-p-tolylphosphino)-1,1′-binaphtyl, (R)-2,2′-bis [di(3,5-xylyl)phosphino]-1,1′-binaphtyl, (S)-2,2′-bis [di(3,5-xylyl)phosphino]-1,1′-binaphtyl, (R)-5,5′-bis(diphenylphosphino)-4,4′-bi-1,3-benzodioxole, (S)-5,5′-bis(diphenylphosphino)-4,4′-bi-1,3-benzodioxole, (R)-5,5′-bis(di [3,5-xylyl]phosphino)-4,4′-bi-1,3-benzodioxole, (S)-5,5′-bis(di [3,5-xylyl]phosphino)-4,4′-bi-1,3-benzodioxole, (R)-5,5′-bis(di [3,5-di-t-butyl-4-methoxyphenyl]phosphino)-4,4′-bi-1,3-benzodioxole, (S)-5,5′-bis(di [3,5-di-t-butyl-4-methoxyphenyl]phosphino)-4,4′-bi-1,3-benzodioxole, (R)-1,13-bis(diphenylphosphino)-7,8-dihydro-6H-dibenzo[f,h][1,5]dioxin, (S)-1,13-bis(diphenylphosphino)-7,8-dihydro-6H-dibenzo[f,h][1,5]dioxin, (R)-2,2′,6,6′-tetramethoxy-4,4′-bis(diphenylphosphino)-3,3′-bipyridine, (S)-2,2′,6,6′-tetramethoxy-4,4′-bis(diphenylphosphino)-3,3′-bipyridine, (R)-2,2′,6,6′-tetramethoxy-4,4′-bis(di [3,5-xylyl]phosphino)-3,3′-bipyridine, (S)-2,2′,6,6′-tetramethoxy-4,4′-bis(di [3,5-xylyl]phosphino)-3,3′-bipyridine, (R)-2,2′-bis(diphenylphosphino)-6,6′-dimethoxy-1,1′-biphenyl, (S)-2,2′-bis(diphenylphosphino)-6,6′-dimethoxy-1,1′-biphenyl, (R)-bis(diphenylphosphino)-4,4′,6,6′-tetramethoxy-1,1′-biphenyl, (S)-bis(diphenylphosphino)-4,4′,6,6′-tetramethoxy-1,1′-biphenyl, (R)-6,6′-bis(diphenylphosphino)-2,2′,3,3′-tetrahydro-5,5′-bi-1,4-benzodioxin, (S)-6,6′-bis(diphenylphosphino)-2,2′,3,3′-tetrahydro-5,5′-bi-1,4-benzodioxin, (R)-(+)-2,2′-bis(diphenylphosphino)-5,5′,6,6′,7,7′,8,8′-octahydro-1,1′-binaphthyl, (S)-(−)-2,2′-Bis(diphenylphosphino)-5,5′,6,6′,7,7′,8,8′-octahydro-1,1′-bi-naphthyl, (R)-5,5′-bis(diphenylphosphino)-2,2,2′,2′-tetrafluoro-4,4′-bi-1,3-benzodioxole, (S)-5,5′-bis(diphenylphosphino)-2,2,2′,2′-tetrafluoro-4,4′-bi-1,3-benzodioxole, (R)-1-[(R)-1-[di(3,5-xylyl)phosphino]ethyl]-2-[2-[di(3,5-xylyl)phosphino]phenyl]ferrocene, (S)-1-[(S)-1-[di(3,5-xylyl)phosphino]ethyl]-2-[2-[di(3,5-xylyl)phosphino]phenyl]ferrocene, (R)-1-[(R)-1-[bis [3,5-bis(trifluoromethyl)phenyl]phosphino]ethyl]-2-[2-(diphenylphosphino)phenyl]ferrocene, and(S)-1-[(S)-1-[bis [3,5-bis(trifluoromethyl)phenyl]phosphino]ethyl]-2-[2-(diphenylphosphino)phenyl]ferrocene.

In some aspects, the ligand is selected from a Segphos ligand, a P (o-tolyl)₃ ligand, a P (m-tolyl)₃ ligand, and a P (p-tolyl)₃. In some aspects, the ligand is a Segphos ligand selected from (R)-Segphos, (R)-DM-Segphos, and (R)-DTBM-Segphos.

In some embodiments, the ligand is selected from a MeO-BIPHEP ligand. In some embodiments, the MeO-BIPHEP ligand is selected from (R)-hexaMeOBIPHEP and (R)-o-An-MeOBIPHEP:

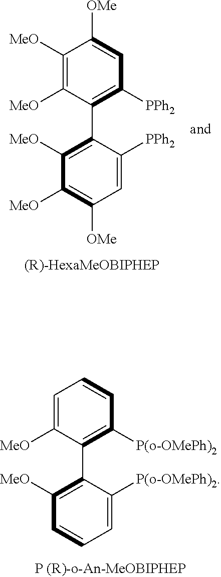

In some embodiments, the MeO-BIPHEP ligand is a GARPHOS ligand.

In some embodiments of the disclosure, the equivalent ratio of the ligand to the transition metal catalyst is suitably 1:1, about 1.1:1, about 1.25:1 or about 1.5:1.

In some aspects, the solvent predominantly comprises at least one non-polar solvent or at least one polar aprotic solvent or a combination of any of the foregoing. In some such aspects, the solvent predominantly comprises toluene, tetrahydrofuran, or 2-methyltetrahydrofuran (2-MeTHF), or a combination of any of the foregoing. In some aspects the solvent system predominantly comprises toluene and tetrahydrofuran or toluene and 2-methyltetrahydrofuran. In such aspects, the volume ratio of toluene to THF or 2-MeTHF is suitably about 90:10, about 75:25, about 60:40, about 50:50, about 40:60, about 25:75, or about 10:90, and any range constructed therefrom, such as from about 90:10 to about 10:90, from about 75:25 to about 25:75, or from about 60:40 to about 40:60.

In some aspects, the base is an organic base. In some such aspects, the organic base is selected from triethylamine, N,N′-diisopropylethylamine, and 1,4-diazabicyclo[2.2. 2]octane. In one aspect, the base is N,N′-diisopropylethylamine. In some embodiments, the base is in stoichiometry excess as compared to compound (1), such as an equivalent ratio of about 1.1:1, about 1.25:1, about 1.5:1, about 1.75:1, about 2:1, or about 2.5:1.

In some embodiments, the equivalent ratio of 2,3-dihydrofuran to compound (1) is suitably about 1.1:1, about 2:1, about 3:1, about 4:1, about 5:1, about 6:1, about 7:1, about 8:1, about 9:1, or about 10:1, and any range constructed therefrom, such as from about 1.1:1 to about 10:1, from about 3:1 to about 8:1, from about 4:1 to about 6:1.

In some embodiments, the reaction temperature is about 30° C., about 35° C., about 40° C., about 50° C., about 60° C., about 70° C., about 80° C., or about 90° C., or reflux, and any range constructed therefrom, such as for instance from about 30° C. to about 90° C., from about 35° C. to about 80° C., from about 40° C. to about 70° C. For instance and without limitation, in some embodiments when the solvent predominantly comprises toluene and 2-methyltetrahydrofuran, the reaction temperature is suitably from about 30° C. to about 70° C., from about 35° C. to about 60° C., or from about 40° C. to about 50° C.

In some embodiments of the disclosure, the reaction product mixture comprising compound (2) may be worked up and optionally isolated such as by, for instance and without limitation, one or more of: filtration; treatment with a metal scavenger: washing with an aqueous phase (e.g., a brine solution); neutralization with an aqueous solution of an acid or base: solvent exchange: phase separation: treatment with an oxidant: crystallization or precipitation by addition of an anti-solvent and optional seed crystal addition and cooling: evaporation: distillation; and drying.

In some aspects, the reaction product mixture may be filtered; the filtrate may be treated with a transition metal scavenger (e.g., an aqueous solution of APDTC (ammonium pyrrolidine dithiocarbamate) to scavenge palladium); the treated filtrate evaporated to form compound (2) residue: the residue may be treated with an oxidant (such as an aqueous solution of TEMPO); and resulting solution of compound (2) may be distilled to form an oil comprising compound (2).

In any of the various aspects of the disclosure, the conversion of compound (1) to compound (2) is at least 70%, at least 75%, at least 80%, or at least 85%.

In any of the various aspects of the disclosure, the purity of compound (2) as measured by high performance liquid chromatography is at least 90 area % or at least 95 area %.

In any of the various aspects of the disclosure, the chiral purity of compound (2) is at least 90 area % or at least 95 area %.

In some aspects of the disclosure, compound (1) is of the structure (1a):

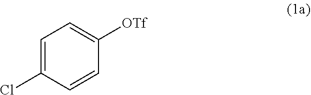 [SMILES: O=S(=O)(Oc1ccc(Cl)cc1)C(F)(F)F]

wherein OTf denotes trifluoromethanesulfonate.

### Preparation of Compound (4)

One aspect of the present disclosure further comprises a process for preparing compound (4). The process comprises step 3: forming a reaction mixture comprising a hydroxylamine solution, a solvent, and compound (3), and reacting the reaction mixture to form a reaction product mixture comprising compound (4) according to the following reaction scheme:

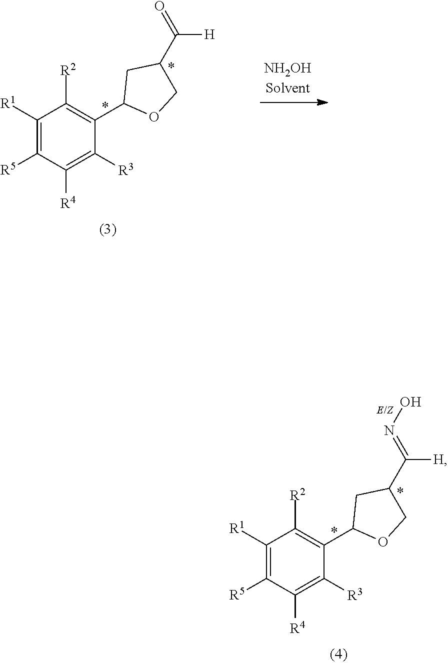

wherein R¹ to R⁵ and the asterisks are as defined elsewhere herein and E/Z denotes E/Z isomers.

In some embodiments, NH₂OH is suitably in an aqueous solution thereof, such as, for instance and without limitation, a 50 wt % aqueous solution. In some embodiments, the equivalent ratio of NH₂OH to compound (3) is suitably about 1.05:1, about 1:1, about 1.15:1, about 1.2:1, about 1.25:1, about 1.3:1, about 1.5:1, about 2:1, or about 2.5:1 and any range constructed therefrom, such as from about 1.05:1 to about 2.5:1, from about 1.1:1 to about 2:1, or from about 1.1:1 to about 1.5:1.

In some aspects, the solvent predominantly comprises at least one non-polar solvent. In some aspects, the solvent predominantly comprises toluene. In some aspects, a solution of compound (3) as described elsewhere herein, such as in a non-polar solvent, such as toluene, is used to form the reaction mixture.

In some embodiments, the reaction temperature is suitably about 5° C., about 10° C., about 20° C., about 30° C., about 40° C., about 50° C., about 60° C., or about 70° C., any range constructed therefrom, such as from about 5° C. to about 70° C., from about 10° C. to about 50° C., or from about 20° C. to about 40° C.

In some embodiments, the reaction product mixture comprising compound (4) may be optionally worked up. In some work-up aspects, the reaction product mixture may be quenched with a brine solution followed by isolation of the organic phase comprising compound (4), such as by phase separation. The isolated organic phase may optionally be washed with water, followed by isolation of the washed organic phase.

In some work-up aspects, the organic phase may be concentrated, such as under vacuum, to form a residue. The residue may be optionally filtered and then dissolved in a non-polar solvent system, such as toluene or the combination of toluene and MTBE. Compound (4) may then be isolated from solution by addition of an anti-solvent, for instance, n-heptane and, optionally, seed crystals of compound (4) to form a slurry comprising compound (4). Compound (4) may then be isolated by filtration or centrifugation. The isolated solids may then be optionally washed, such as with toluene/n-heptane, and dried.

In any of the various aspects of the disclosure, the conversion of compound (3) to compound (4) is at least 70%, at least 75%, at least 80%, or at least 85%.

In any of the various aspects of the disclosure, the purity of compound (4) as measured by high performance liquid chromatography is at least 90 area %, at least 95 area %, or at least 98 area %.

In any of the various aspects of the disclosure, the chiral purity of compound (4) is at least 90 area % or at least 95 area %.

In some aspects of the disclosure, compound (4) is of the structure (4a):

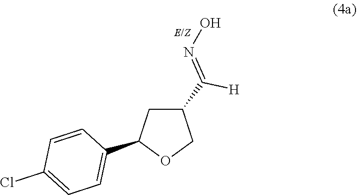 [SMILES: O/N=C/[C@@H]1CO[C@@H](c2ccc(Cl)cc2)C1]

wherein E/Z denotes E/Z isomers.

### First Route for Preparation of Compound (6)

One aspect of the present disclosure further comprises a process for preparing compound (5) from compound (4) and for preparing compound (6) from compound (5).

The process for preparing compound (5) comprises step 4a: forming a reaction mixture comprising compound (4), a reagent, and a solvent, and reacting the reaction mixture to form a reaction product mixture comprising compound (5) according to the following reaction scheme:

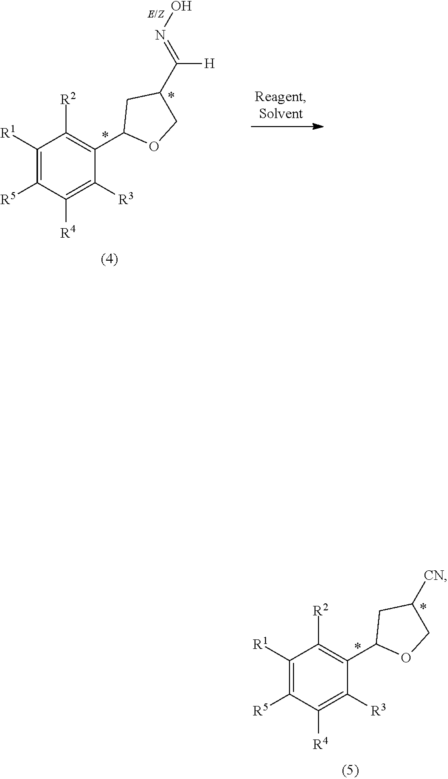

wherein R¹ to R⁵, the asterisks, and E/Z are as defined elsewhere herein.

In some aspects, the reagent is a dehydrating reagent. Non-limiting examples of reagents include carbonyldiimidazole, acetic anhydride, trifluoroacetic anhydride, T3P (1-propanephosphonic anhydride), EDCI (N-ethyl-N′-(3-dimethylaminopropyl) carbodiimide hydrochloride), DIC (N,N′-diisopropylcarbodiimide), PyCloP (chlorotripyrrolidinophosphonium hexafluorophosphate), Ac₂O, POCl₃, SOCl₂, Na₂CO₃, Burgess reagent, CH₃SO₂O, DBU, DCM, CH₃SOCl₂, Et;N, and CuOAc₂. In some aspects, the reagent is carbonyldiimidazole.

In some embodiments, the equivalent ratio of the reagent to compound (4) is suitably about 1.05:1, about 1.1:1, about 1.2:1, about 1.25:1, about 1.3:1, about 1.4:1, about 1.5:1, or about 2:1, and any range constructed therefrom, such as for instance from about 1.05:1 to about 2:1, from about 1.1:1 to about 1.5:1, or from about 1.2:1 to about 1.3:1.

In some aspects, the solvent predominantly comprises at least one non-polar solvent or at least one polar solvent or a combination of any of the foregoing. In some aspects, the solvent is selected from a C₁₋₆ alcohol, a C₁₋₆ ester, and an ether, or a combination of any of the foregoing. In some aspects, the solvent predominantly comprises MTBE, ethyl acetate, ethanol, methanol, i-propanol, THF, or 2-MeTHF, or a combination of two or more thereof. In some aspects, the solvent predominantly comprises MTBE. The reaction mixture comprises compound (4) in solution in the solvent.

In some aspects, the reaction product mixture comprising compound (5) in solution may be directly used without isolation for conversion to compound (6).

In some aspects, the reaction product mixture comprising compound (5) in solution may be worked up. Compound (5) may be worked up such as by, for instance and without limitation, one or more of: filtration: washing with an aqueous phase: neutralization by contact with an aqueous acid or base: solvent exchange; phase separation: crystallization or precipitation by addition of an anti-solvent and optional seed crystal addition and cooling: evaporation: distillation; and drying.

In some work-up aspects, the reaction product mixture may be water washed by combining the reaction product mixture with water followed by phase separation to isolate the organic phase comprising compound (5). The reaction product mixture or isolated washed organic phase may be washed with an aqueous acid solution followed by phase separation and isolation of the organic phase. In some aspects, the acid is a weak acid such as, for instance and without limitation, citric acid. The acid wash may optionally be followed by a water wash followed by phase separation and isolation of the organic phase. Water wash and aqueous neutralization steps may be followed by drying, such as for instance and without limitation, with a drying agent (e.g., Na₂SO₄ or MgSO₄). The reaction product mixture or any of the organic phases may be optionally filtered. In some aspects, the reaction product mixture or the final processed organic phase comprising compound (5) may be concentrated to an oil comprising compound (5) in solution, and used directly to form compound (6). Concentration may be done by methods known in the art such as by distillation or evaporation. In some aspects, compound (6) may be isolated, such as, for instance and without limitation, by anti-solvent addition, optional seed crystal addition, cooling, and filtration.

In some aspects, compound (5) is of the structure (5a):

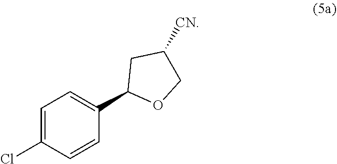 [SMILES: N#C[C@@H]1CO[C@@H](c2ccc(Cl)cc2)C1]

The process for preparing compound (6) comprises step 5a: forming a reaction mixture comprising compound (5), hydroxylamine, and a polar solvent, and reacting the reaction mixture to form a reaction product mixture comprising compound (6) according to the following reaction scheme:

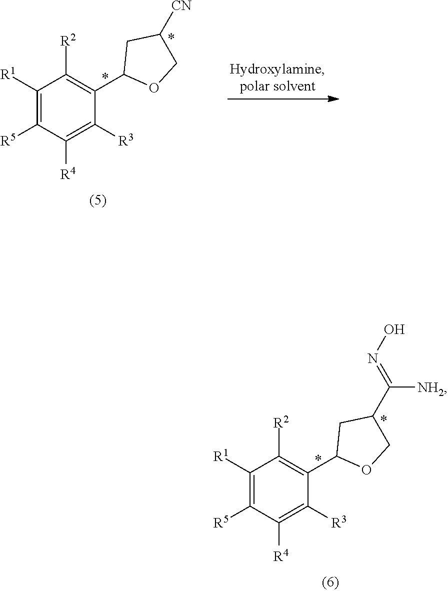

wherein R¹ to R⁵ and the asterisks are as defined elsewhere herein.

In some embodiments, the equivalent ratio of hydroxylamine to compound (5) is suitably about 1.1:1, about 1.2:1, about 1.3:1, about 1.4:1, about 1.5:1, about 1.6:1, about 1.7:1, about 1.8:1, about 1.9:1, about 2:1, about 2.5:1, or about 3:1, and any range constructed therefrom, such as for instance from about 1.1:1 to about 3:1, from about 1.2:1 to about 2:1, or from about 1.3:1 to about 1.7:1.

In some aspects, the solvent predominantly comprises at least one polar protic solvent. In some aspects, the solvent predominantly comprises a C₁₋₅ alcohol. In some aspects, the solvent predominantly comprises t-amyl alcohol, i-propyl alcohol, methanol, or ethanol. In some aspects, the solvent predominantly comprises i-propyl alcohol. In some aspects, the solvent predominantly comprises t-amyl alcohol.

The reaction product mixture comprising compound (6) in solution may be worked up. Compound (6) may be worked up such as by, for instance and without limitation, one or more of: filtration: washing with an aqueous phase; neutralization by contact with an aqueous acid or base: solvent exchange: phase separation: crystallization or precipitation by addition of an anti-solvent and optional seed crystal addition and cooling: evaporation: distillation; and drying.

In some aspects, the reaction product mixture may be combined with water and compound (6) seed crystals and cooled to form a slurry of compound (6). Compound (6) may be isolated from the slurry by filtration or centrifugation. Isolated compound (6) may be optionally washed with an anti-solvent, such as n-heptane, and then dried.

In any of the various aspects of the disclosure, the yield of compound (6) based on compound (4) is at least 75%, at least 80%, or at least 85%.

In some aspects, compound (6) is of the structure (6a):

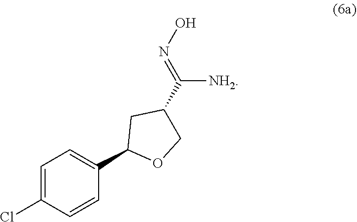 [SMILES: N/C(=N\O)[C@@H]1CO[C@@H](c2ccc(Cl)cc2)C1]

### Second Route for Preparation of Compound (6)

One aspect of the present disclosure further comprises a process for preparing compound (7) from compound (4) and for preparing compound (6) from compound (7).

The process for preparing compound (5) comprises step 4b: forming a reaction mixture comprising compound (4), an oxidant, an acid, and a solvent, and reacting the reaction mixture to form a reaction product mixture comprising compound (7) according to the following reaction scheme:

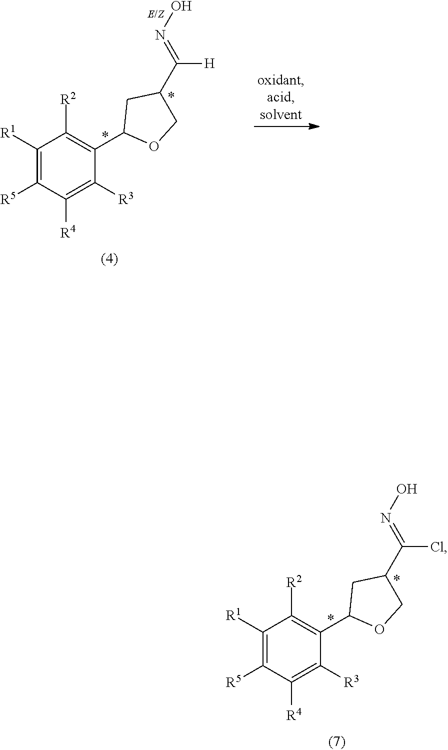

wherein R¹ to R⁵, the asterisks, and E/Z are as defined elsewhere herein.

In some aspects, the oxidant is as disclosed elsewhere herein. In some aspects, the oxidant is selected from N-chlorosuccinimide, t-BuOCl, chloramine-T, NaOCl, 1.3 dichloro-5,5-dimethylhydantoin, N-chlorophthalimide, and 2-chlorobenzo[d]isothiazole-3(2H) one 1,1 dioxide. In one aspect, the oxidant is N-chlorosuccinimide. In some embodiments, the equivalent ratio of the oxidant to compound (4) is suitably about 1.01:1, about 1.05:1, about 1.1:1, about 1.15:1, about 1.2:1, about 1.3:1, about 1.4:1, about 1.5:1, or about 2:1, and any range constructed therefrom, such as for instance from about 1.01:1 to about 2:1, from about 1.01:1 to about 1.5:1, or from about 1.05:1 to about 1.2:1.

In some aspects, the acid is an inorganic acid, such as for instance and without limitation, HCl, H₂SO₄, HNO₃, or H₃PO₄. In some aspects the acid is concentrated acid, such as concentrated aqueous acid. In one aspect, the acid is aqueous concentrated HCl. In some embodiments, the equivalent ratio of the acid to compound (4) is suitably about 0.01:1, about 0.05:1, about 0.1:1, about 0.15:1, about 0.2:1, about 0.3:1, about 0.4:1, or about 0.5:1, and any range constructed therefrom, such as for instance from about 0.01:1 to about 0.5:1, from about 0.05:1 to about 0.3:1, or from about 0.05:1 to about 0.2:1.

In some aspects, the solvent predominantly comprises at least one non-polar solvent or at least one polar solvent or a combination of any of the foregoing. In some aspects, the solvent predominantly comprises MTBE, EtOAc, DMF, DCM, MeOH, ACN, toluene, IPAc, or DMF, or a combination of any of the foregoing. In some aspects, the solvent predominantly comprises MTBE. In some aspects, the solvent predominantly comprises EtOAc. The reaction mixture comprises compound (4) in solution in the solvent.

In some aspects, the reaction product mixture comprising compound (7) in solution may be directly used without isolation for conversion to compound (6). In some embodiments, compound (7) is in solution in an organic phase.

In some aspects, the reaction product mixture comprising compound (7) in solution may be worked up and optionally isolated. Compound (7) may be worked up such as by, for instance and without limitation, one or more of: filtration; washing with an aqueous phase; neutralization by contact with an aqueous acid or base; solvent exchange; phase separation; crystallization or precipitation by addition of an anti-solvent and optional seed crystal addition and cooling; evaporation; distillation; and drying.

In some aspects, compound (7) is compound (7a) of the following structure:

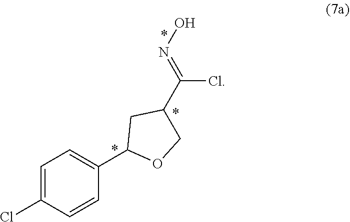 [SMILES: O/N=C(\Cl)C1COC(c2ccc(Cl)cc2)C1]

The process for preparing compound (6) comprises step 5b: forming a reaction mixture comprising compound (7) ammonia, and a polar solvent, and reacting the reaction mixture to form a reaction product mixture comprising compound (6) according to the following reaction scheme:

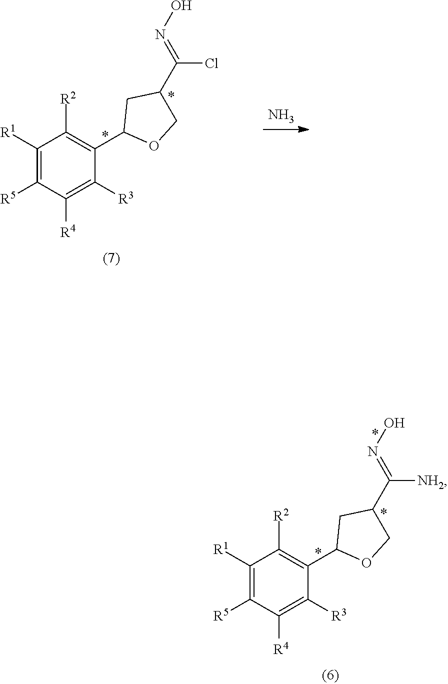

wherein R¹ to R⁵ and the asterisks are as defined elsewhere herein.

In some aspects, the solvent predominantly comprises at least one non-polar solvent or at least one polar solvent or a combination of any of the foregoing. In some aspects, the solvent predominantly comprises ethyl acetate or MTBE. In some aspects, the solvent predominantly comprises ethyl acetate. In some aspects, the solvent predominantly comprises MTBE. The reaction mixture comprises compound (7) in solution in the solvent.

In some aspects, the ammonia is in solution in a C₁₋₄ alcohol. In some such aspects, the ammonia is in solution in methanol.

In some aspects, the reaction product mixture comprising compound (6) in solution may be worked up and optionally isolated. Compound (6) may be worked up such as by, for instance and without limitation, one or more of: filtration; washing with an aqueous phase: neutralization by contact with an aqueous acid or base: solvent exchange: phase separation: crystallization or precipitation by addition of an anti-solvent and optional seed crystal addition and cooling: evaporation; distillation; and drying.

In some work-up aspects, the reaction product mixture may be water washed by combining the reaction product mixture with water followed by phase separation to isolate the organic phase comprising compound (6). The washed organic phase may be dried, such as for instance and without limitation, with a drying agent (e.g., Na₂SO₄ or MgSO₄). In some aspects, the dried organic phase is treated with charcoal and then filtered. The organic phase may be optionally concentrated by evaporation or distillation. The optionally concentrated organic phase may then be combined with an anti-solvent (e.g., n-heptane) and optional seed crystals. In some aspects, the resulting mixture may be concentrated by evaporation or distillation and cooled to form a slurry of compound (6). Compound (6) may isolated from the slurry by filtration or centrifugation, and the collected solids may then be optionally dried.

In any of the various aspects of the disclosure, the yield of compound (6) based on compound (4) is at least 75%, at least 80%, or at least 85%.

### Compounds (3), (4), and (7)

Some aspects of the disclosure are directed to compound (3) of the following structure, or a salt thereof:

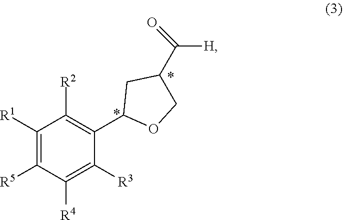

wherein R¹ to R⁵ and the asterisks are as defined elsewhere herein.

Some aspects of the disclosure are directed to the following compound (3) species, compound (3a), or a salt thereof:

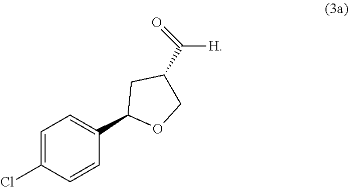

Some aspects of the disclosure are directed to compound (4) of the following structure, or a salt thereof:

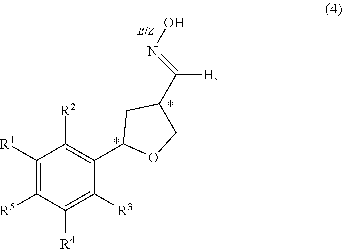

wherein R¹ to R⁵, the asterisks, and E/Z are as defined elsewhere herein.

Some aspects of the disclosure are directed to the following compound (4) species, compound (4a), or a salt thereof:

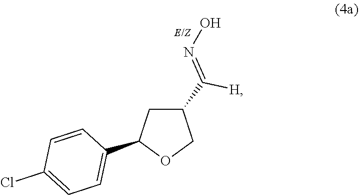

wherein E/Z is as defined elsewhere herein.

Some aspects of the disclosure are directed to compound (7) of the following structure, or a salt thereof:

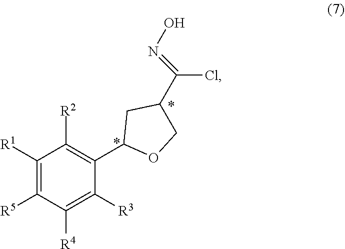

wherein R¹ to R⁵ and the asterisks are as defined elsewhere herein.

Some aspects of the disclosure are directed to the following compound (7) species, compound (7a), or a salt thereof:

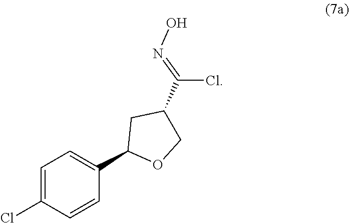 [SMILES: O/N=C(\Cl)[C@@H]1CO[C@@H](c2ccc(Cl)cc2)C1]

### Integrated Process Schemes

Some aspects of the disclosure relate to an overall process for preparing compound (4) according to steps 1 to 3 as follows:

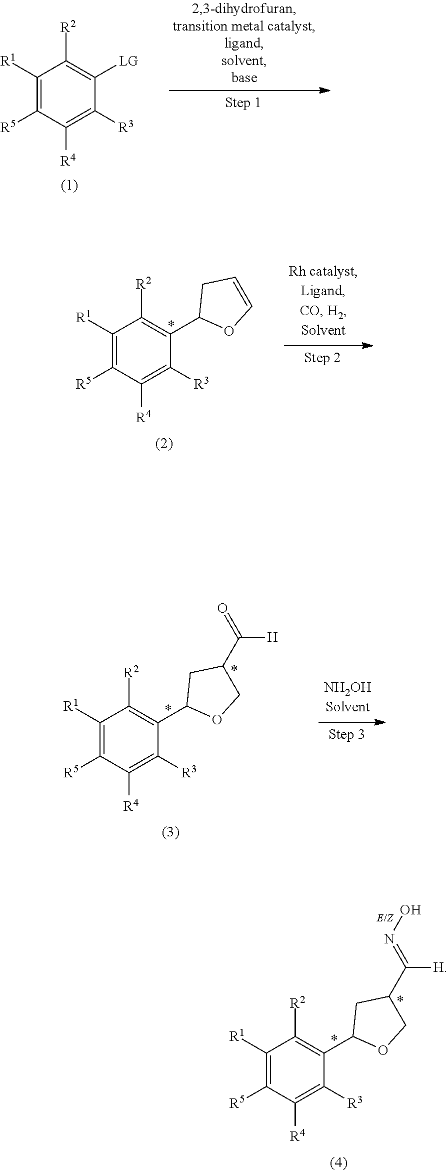

In the scheme of steps 1 to 3, R¹ to R⁵; LG; the asterisks; E/Z; the step 1 transition metal catalyst, ligand, solvent, base, and reaction conditions; the step 2 Rh catalyst, ligand, solvent, and reaction conditions, and the step 3 solvent and reaction conditions are as described elsewhere herein. In some aspects, compound (3) is not isolated prior to step 3. In some aspects, compound (1) is the species of compound (1a), compound (2) is the species of compound (2a), compound (3) is the species of compound (3a), and compound (4) is the species of compound (4a), each as described elsewhere herein.

Some aspects of the disclosure relate to an overall process for preparing compound (6) according to steps 1-3 above and further including steps 4a and 5a as follows:

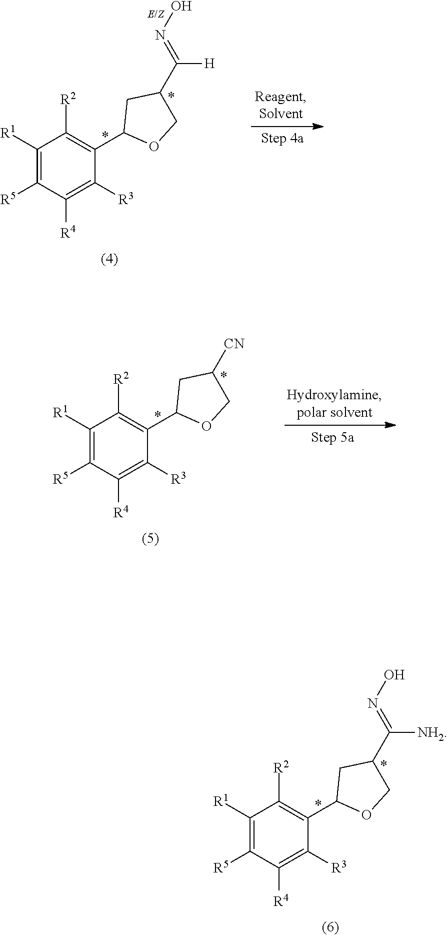

In the scheme of steps 4a and 5a, R¹ to R⁵; the asterisks; E/Z; the step 4a reagent and solvent; and the step 5a polar solvent are as described elsewhere herein. In some aspects, compound (5) is not isolated prior to step 5a. In some aspects, compound (4) is the species of compound (4a), compound (5) is the species of compound (5a), and compound (6) is the species of compound (6a), each as described elsewhere herein.

Some aspects of the disclosure relate to an overall process for preparing compound (6) according to steps 1 to 3 above and further including steps 4b and 5b as follows:

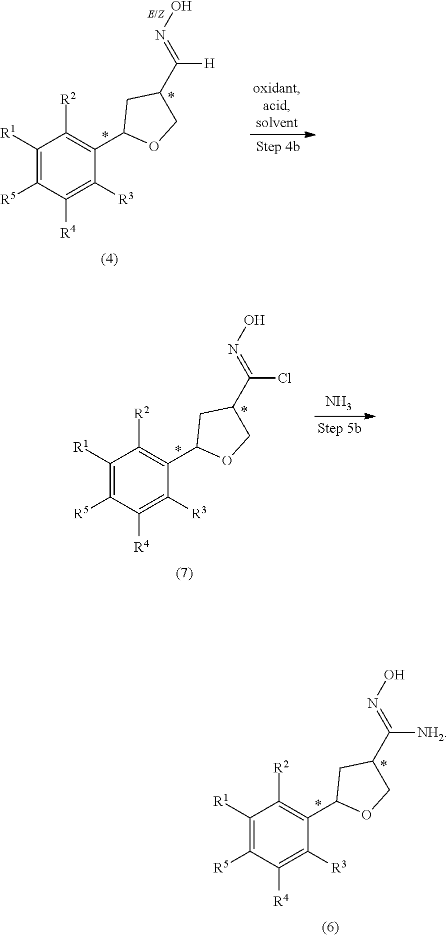

In the scheme of steps 4b and 5b, R¹ to R⁵; the asterisks; E/Z; and the step 4b oxidant, acid, and solvent are as described elsewhere herein. In some aspects, compound (7) is not isolated prior to step 5b. In some aspects, compound (4) is the species of compound (4a), compound (7) is the species of compound (7a), and compound (6) is the species of compound (6a), each as described elsewhere herein.

### EXAMPLES

### Example 1. Preparation of compound (2a) via Heck Arylation

Compound (2a)((R)-2-(4-chlorophenyl)-2,3-dihydrofuran) was prepared from compound (1a)(4-chlorophenyl trifluoromethanesulfonate) according to the following reaction step 1′.

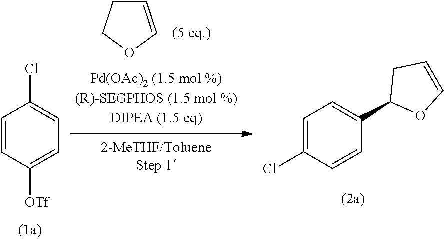 [SMILES: C1=COCC1 ; O=S(=O)(Oc1ccc(Cl)cc1)C(F)(F)F ; Clc1ccc([C@H]2CC=CO2)cc1]

To a solution of compound (1a)(336 g, 1.29 mol, 1 equiv) in a mixture of 2-MeTHF (840 mL) and toluene (840 mL) was charged DIPEA (336 mL, 1.93 mol, 1.5 equiv). The solution was sparged with N₂ for 30 minutes at room temperature before Pd(OAc)₂(6.38 g, 19.4 mmol, 0.015 equiv), (R)-SEGPHOS (11.72 g, 19.4 mmol, 0.015 equiv), and 2,3-dihydrofuran (6.45 mol, 5.0 equiv) were charged sequentially. The suspension was then sparged with N₂ at room temperature (rt) for 30 minutes before the reactor was warmed to 45° C. and stirred for 20 h at this temperature. The reactor was then cooled to rt and filtered over a pad of Celite. To the resulting filtrate was added aqueous APDTC (0.02 wt % in 336 mL water) and the suspension stirred for 5 h at 25° C. The layers were then separated and the organic phase concentrated to dryness. To the resulting residue was added TEMPO (2 wt %) and the resulting solution distilled under vacuum (oil bath, 110° C., 5 Torr) to obtain a colorless oil (194 g, 82%, 95.4% HPLC purity, 96.5% chiral purity). 1H NMR (400 MHZ, DMSO-d₆) δ 7.48-7.28(m, 5H), 6.57(q, J=2.5 Hz, 1H), 5.51(dd, J=10.7, 8.1 Hz, 1H), 4.98 (q, J=2.5 Hz, 1H), 3.39(dd, J=3.2, 1.7 Hz, 1H), 3.05(ddt, J=15.4, 10.7, 2.4 Hz, 1H), 2.42(ddt, J=15.3, 8.1, 2.4 Hz, 1H). 13C NMR (101 MHZ, DMSO-d₆) δ 145.76, 142.50, 132.52, 128.84, 127.74, 99.39, 81.05, 37.84. HRMS: calculated for C₁₀H₁₀ClO [M+H]⁺: 181.0420, found: 180.0419.

### Example 2. Preparation of compound (3a)((3S,5R)-5-(4-chlorophenyl)tetrahydrofuran-3-carbaldehyde) via hydroformylation

Compound (3a) was prepared from compound (2a) according to the following reaction step 2′.

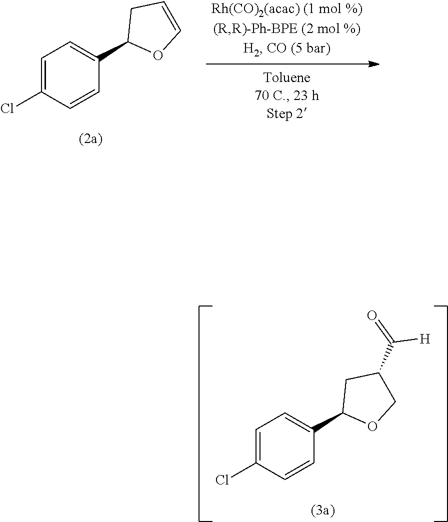

Preparation of the catalyst solution. In a glovebox (<1 ppm 02) a 250 mL flask was charged with Rh(CO)₂(acac)(1.71 g, 0.007 mol, 0.010 equiv) and dissolved in 40 ml of toluene. (R,R)-Ph-BPE (6.73 g, 0.013 mol, 0.020 equiv) was slowly added (strong gas development) and the weighting bottle and flask neck were rinsed with 20 ml of toluene. A yellow solution was obtained after stirring for 15 min. The solution was transferred to an addition vessel, and the flask was rinsed twice with 10 ml of toluene used. The addition vessel was closed and removed from the glovebox.

Preparation of the reaction mixture. In air, a 2000 ml autoclave was charged with compound (2a)(120 g, 98.36 mL, 0.664 mol, 1 equiv), and dissolved in toluene, (degassed, 69.2 g, 80 mL, 1.13 equiv). The autoclave was sealed. The addition vessel containing catalyst was connected to the autoclave. The autoclave and line to the addition vessel were purged 3× with 7 bar of Argon. The catalyst solution was then added to the autoclave with Argon pressure. The addition vessel was rinsed with toluene (degassed, 17.3 g, 20 mL, 0.283 equiv) into the autoclave. After everything had been placed into autoclave, the autoclave was purged 3× with 10 bar of H₂/CO and the relative working pressure was set to 5 bar working pressure. The autoclave contents were was heated to 70° C. and stirred at 1200 rpm. After 22 h the autoclave was cooled to rt and the pressure was released yielding a crude solution of compound (3a). Testing indicated 89.1% conversion of compound (2a)(compound (3a) contained 3.4% regioisomer). The solution of crude compound (3a) was telescoped to the next step without workup. 1H NMR (400 MHZ, CDCl₃) δ 9.78 (d, J=1.8 Hz, 1 H), 7.29-7.24(m, 4H), 4.95-4.89(m, 1H), 4.27-4.19(m, 2H), 3.29-3.16(m, 2H), 2.69(ddd, J=12.9, 6.8, 4.2 Hz, 1H), 2.05-1.96(m, 1H). 13C NMR (101 MHZ, DMSO-d₆) δ 202.78, 141.84, 132.26, 128.70, 127.99, 79.30, 67.28, 51.47, 34.98. HRMS calculated for C₁₁H₁₂ClO₂:211.0526, found: 211.0523.

### Example 3. Preparation of oxime compound (4a)((E)-5-(4-chlorophenyl)tetrahydrofuran-3-carbaldehyde oxime) via hydroxylamine addition

Compound (4a) was prepared from compound (3a) according to the following reaction step 3″.

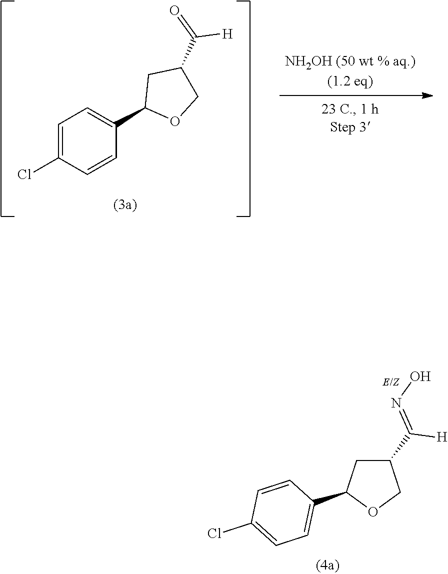

A 2500 ml 4-neck sulfonation flask was charged with a solution of crude compound (3a)(139.88 g, 0.664 mol, 1 equiv) solved in toluene (1.03 kg, 1.2 L, 16.9 equiv). To the stirred solution was added hydroxylamine solution (50% in water, 51.91 g, 48.15 mL, 0.797 mol, 1.2 equiv) over the course of 30 minutes. After stirring for an additional 80 minutes, the reaction was quenched with half saturated brine (200 mL) and stirred for 20 minutes. The layers were separated and the organic phase was washed with water (200 g, 200 mL, 16.72 equiv). The orange organic phase was concentrated in vacuo (40° C., 100-70 mbar) to a residue (445.95 g). The orange residue was filtered over a silica pad (240 g silica), which was washed with MTBE (884.7 g, 1.2 L, 15.11 equiv). The yellow filtrate was concentrated in vacuo (40° C., 220-10 mbar) to provide crude compound (4a)(155.80 g), which was then dissolved in toluene (519 g, 600 mL, 8.48 equiv) at rt. To the solution was added n-heptane (256.5 g, 375 mL, 3.86 equiv) over the course of 10 minutes. To the yellow solution, compound (4a) seed crystals (1.00 g, 0.004 mol, 0.007 equiv) were added at the mixture was stirred for 60 min at rt. To the resulting suspension was added n-heptane (769.5 g, 1.13 L, 11.57 equiv) over the course of 2 h. The suspension was stirred for 17 h before being filtered. The flask and wet cake were washed twice with a 1:1 v/v mixture of toluene (86.5 g, 100 mL, 1.41 equiv) and n-heptane (68.4 g, 100 mL, 1.03 equiv). The solids were dried at 40° C., 10 mbar for 4 h to afford compound (4a) as light yellow crystals (110.8 g, 72.8% yield, 98.4% purity). 1H NMR (400 MHZ, CDCl₃) δ 8.69 (s, 0.1 H minor isomer), 8.24 (s, 1 H OH, major isomer), 7.46(d, J=6.7 Hz, 1H, Major isomer), 7.34-7.19 (m, 4H), 6.81 (d, J=6.2 Hz, 0.1 H, minor isomer), 5.02 (t, J=7.0 Hz, 1H), 4.26 (dd, J=8.8, 7.3 Hz, 1H), 3.86 (dd, J=8.8, 6.8 Hz, 1H), 3.21-3.15 (m, 1H), 2.37 (ddd, J=12.8, 7.3, 6.1 Hz, 1H), 2.06 (ddd, J=12.7, 8.4, 6.7 Hz, 1H). 13C NMR (101 MHZ, CDCl₃) δ 151.51, 140.97, 133.13, 128.58, 126.88, 79.63, 79.63, 71.05, 39.39, 38.64. HRMS: calculated for: C₁₁H₁₃ClNO₂ 226.0635, found: 226.0629.

### Example 4

Compound (5a)((3R,5R)-5-(4-chlorophenyl)tetrahydrofuran-3-carbonitrile) was prepared from compound (4a) according to the following reaction step 4a′ and compound (6a)((3R,5R,Z)-5-(4-chlorophenyl)-N′-hydroxytetrahydrofuran-3-carboximidamide) was prepared from compound (5a) according to the following reaction step 5a.

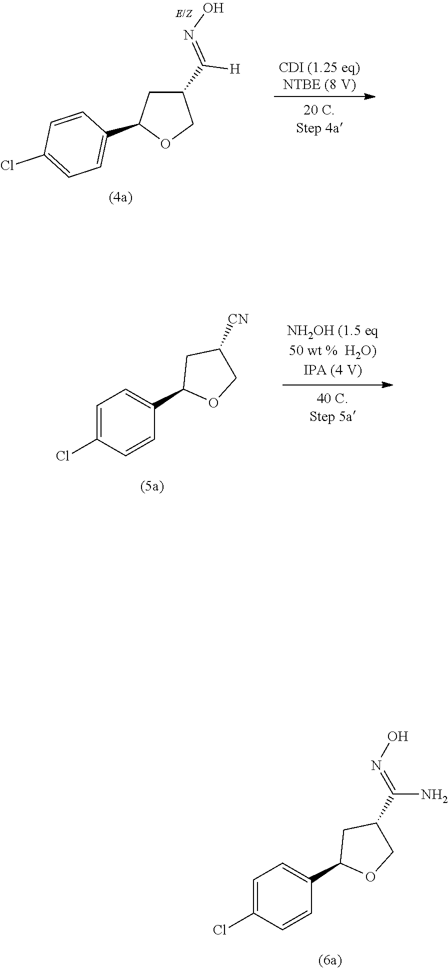 [SMILES: O/N=C/[C@@H]1CO[C@@H](c2ccc(Cl)cc2)C1 ; N#C[C@@H]1CO[C@@H](c2ccc(Cl)cc2)C1 ; N/C(=N\O)[C@@H]1CO[C@@H](c2ccc(Cl)cc2)C1]

In step 4a′, a glass reactor under nitrogen gas was charged with compound (4a)(30.0 g, 123 mmol, 1.0 equiv, E/Z 2:1 ratio) and MTBE (240 mL, 8 V) giving a solution. The internal temperature was adjusted to 25° C. and solid 1,1-carbonylimidazole (24.9 g, 154 mmol, 1.25 equiv) was added in portions (exotherm observed, significant off gassing), and the reaction was left to stir overnight at 25° C. The reaction was sampled for IPC (Target (HPLC): compound (4a)≤1.0% area, Result: 0% area, Met). Water (150 mL, 5V) was added and phases were separated. The organic phase containing compound (5a) was washed with citric acid (150 mL, 5 V, 5 wt % aqueous solution) followed by water (150 mL, 5V) wash. The organic phase was dried with NaSO₄ and filtered. In step 5a′, the organic phase containing compound (5a) was concentrated to oil before addition of isopropyl alcohol (105 mL, 4V). The temperature was adjusted to 40° C. and hydroxylamine (11.6 mL, 190 mmol, 1.5 equiv, 50 wt % of aqueous solution) was charged over 1 h. The reaction was agitated for 12 h. The reaction was sampled for IPC ((Target (HPLC): compound (5a)≤1.0% area, Result: 0% area, Met). Water (26 mL, 1V) was charged and stirred for 30 min. Reaction mixture was cooled to 25° C., seeds were added (2 wt %) and reaction mixture was held for 1 h. Then water (184 mL, 7 V) was added over 1 h and then the temperature was adjusted to 0° C. over 6 h. The temperature was then adjusted to 40° C. held for 3 h and cooled over 6 h to 0° C. The slurry was filtered and the cake was washed with heptane (30 mL, 1V) three times. The solids were dried at room temperature to yield 27.5 g of white solid compound (6a)(80%).

### Example 5

Example 4 was repeated with a variety of ligands.

A reaction using the BPE catalyst

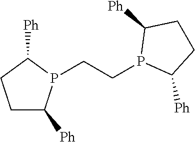 [SMILES: c1ccc([C@H]2CC[C@H](c3ccccc3)P2CCP2[C@@H](c3ccccc3)CC[C@@H]2c2ccccc2)cc1]

provided a conversion of compound (2a) to compound (3a) of 90% to 95%.

A reaction using the bisdiazaphos ligand

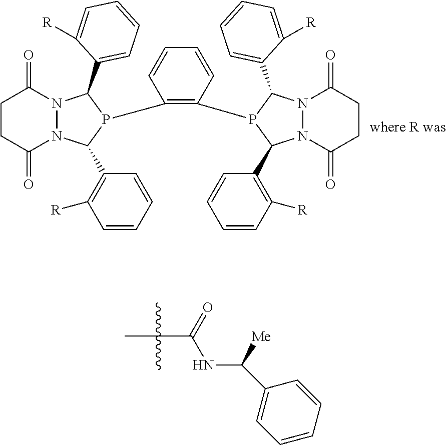

provided a conversion of compound (2a) to compound (3a) of >99%.

A reaction using the indolphos ligand

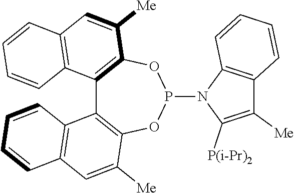

provided a conversion of compound (2a) to compound (3a) of >99%.

A reaction using the ferrocene ligand

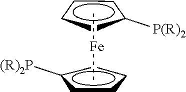

where R was phenyl provided a conversion of compound (2a) to compound (3a) of >99%. A reaction using that ferrocene ligand where R was i-Pr provided a conversion of compound (2a) to compound (3a) of >99%.

A reaction using the xanthene ligand

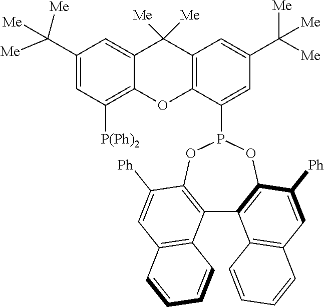

provided a conversion of compound (2a) to compound (3a) of >99%.

### Example 6

Compound (5a) was prepared from compound (4a) according to the following reaction step 4a″ and compound (6a) was prepared from compound (5a) according to the following reaction step 5a″.

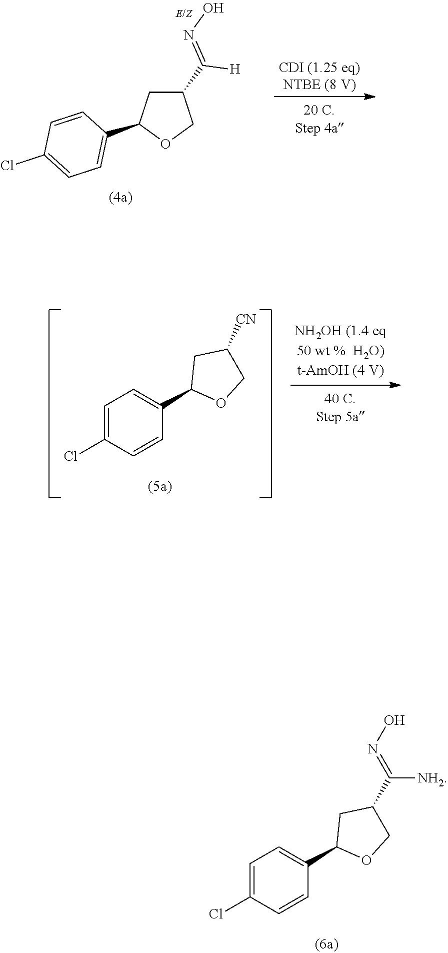

In step 4a″, a glass reactor (R¹) under nitrogen gas was charged with compound 4a (100.0 g, 425 mmol, 1.0 equiv., E/Z 2:1 ratio) and MTBE (400 mL, 4 V) giving a solution. The internal temperature was adjusted to 25° C. A second glass reactor (R²) was charged with solid 1,1-carbonylimidazole (88.0 g, 532 mmol, 1.25 equiv.) and MTBE (400 mL, 4 V) creating slurry. The contents of R² were vacuum transferred to R1 in portions to keep Ti<35° C. (exotherm observed, significant off gassing), and the reaction was left to stir overnight at 25° C. The reaction was sampled for IPC ((Target (HPLC): compound 4a≤1.0% area, Result: 0% area, Met). Water (500 mL, 5 V) was added and the phases were separated. The organic phase containing compound 5a was washed with citric acid (500 mL, 5 V, 5 wt % aqueous solution) followed by water (500 mL, 5V) wash. The organic phase was filtered (solids were discarded). The batch was split into two equal portions by mass (2×44 g, based on the max theoretical yield). The organic phase containing compound 5a was concentrated to 4 V using distillation. Thereafter, continuous distillation was used to perform solvent exchange from MTBE (176 mL, 4 V) to t-AmOH (176 mL, 4 V) until MTBE <1 wt % was achieved. In step 5a″, the temperature was adjusted to 40° C. and hydroxylamine (17.6 mL, 288 mmol, 1.4 equiv., 50 wt % of aqueous solution) was charged over 6 h. The reaction was agitated for 16 h. The reaction was sampled for IPC ((Target (HPLC): compound 5a≤1.0% area, Result: 0% area, Met). The reaction mixture was cooled to 25° C., compound 6a seeds were added (0.2 g, 0.5 wt %) and the reaction mixture was held for 0.5 h to allow seed bed to grow. Heptane (440 mL, 10 V) was charged over 3 h and stirred for 30 min. The temperature was adjusted to 0° C. over 3 h and stirred overnight. The slurry was filtered and the cake was washed with heptane (44 mL, 1V) three times. The solids were dried at 23° C. to yield 43.9 g of white solid compound 6a (85% corrected yield calculated for half of the initial batch).

### Example 7

Compound (7a)((3S,5R,Z)-5-(4-chlorophenyl)-N-hydroxytetrahydrofuran-3-carbimidoyl chloride) was prepared from compound (4e) according to the following reaction step 4b′ and compound (6a) was prepared from compound (7a) according to the following reaction step 5b′.

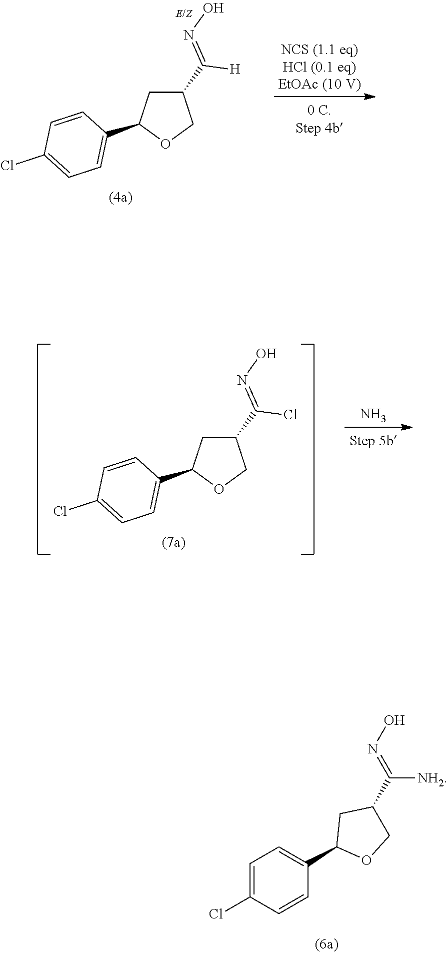

In step 4b′, a glass reactor under nitrogen gas was charged with compound (4a)(5.0 g, 22.2 mmol, 1.0 equiv, E/Z 2:1 ratio) and EtOAc (50 mL, 10 V) giving a suspension. The reaction was stirred at room temperature until full dissolution was achieved. The internal temperature was adjusted to 0° C. and solid N-chlorosuccinimide (3.2 g, 24.4 mmol, 1.1 equiv) was added in portions (small exotherm observed, no color change of the reaction mixture), giving a white suspension. Concentrated aqueous HCl (0.2 g, 2.2 mmol, 0.1 equiv) was added dropwise (reaction mixture changed color to blue) and the reaction was left to stir for 16 h at 0° C. The reaction was sampled for IPC ((Target (HPLC): compound (4a) ≤1.0% area, Result: 0% area). In step 5b′, a solution of NH₃ in MeOH (9.5 mL, 66.5 mmol, 2 M solution, 3.0 equiv) was added slowly to keep the Ti<5° C. The reaction was stirred for 30 min. The reaction was sampled for IPC (Target (HPLC): compound (7a)≤1.0% area, Result: 0% area). The reaction mixture was warmed up to 10° C. Water (15 mL, 3V) was charged and stirred for 30 min. The temperature was raised to 20° C. The organic phase was washed with water twice (15 mL, 3V) more before it was dried over MgSO₄. The cake was washed with additional EtOAc (10 mL, 2 V). The combined organic phase containing compound 6(a) was then subjected to charcoal treatment (20 wt %) then filtered. The cake was washed with EtOAc (10 mL, 2 V). The solution of compound (6a) in EtOAc (70 mL, 14 V) was concentrated to 4 V. The temperature was adjusted to 40° C. before heptane (20 mL, 4 V) was added slowly. Compound (6a) seed crystals (0.2 wt %) were added and that the slurry was aged for 30 min. Distillation was then done until about 3 V of the solvent was removed. The slurry was cooled down to 25° C. over 1 h and then filtered. The collected cake was washed with EtOAc: heptane mixture (1:3) and then dried at room temperature to yield 3.47 g of white solid (65%). 1H NMR (400 MHZ, DMSO)δ 8.98 (s, 1H), 7.41-7.31 (m, 4H), 5.44 (s, 2H), 4.96 (t, J=7.0 Hz, 1H), 4.15 (t, J=7.9 Hz, 1H), 3.84 (dd, J=8.3, 7.4 Hz, 1H), 2.99-2.90 (m, 1H), 2.49-2.45 (m, 1H), 1.87 (ddd, J=12.4, 8.7, 6.6 Hz, 1H). 13C NMR (101 MHZ, DMSO)_{8 152.7, 142.8, 131.9, 128.6, 127.9, 79.3, 71.1, 40.7, 38.1}. HRMS calculated for C₁₁H₁₄CIN₂O₂ [M+H]⁺ 241.0738, found 241.0737.

This written description uses exemplary examples to describe the disclosure, including the best mode, and also to enable any person skilled in the art to practice the disclosure, including making and using any devices or systems and performing any incorporated methods and processes. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A process for preparing compound (3) 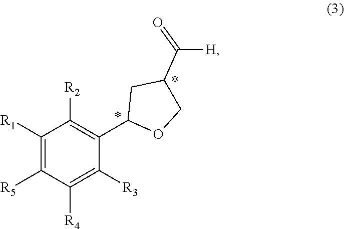
the process comprising forming a reaction mixture comprising CO, H₂, a rhodium catalyst, a ligand, a solvent, and compound (2), and reacting the reaction mixture to form a reaction product mixture comprising compound (3),
wherein:
(i) compound (2) is of the structure 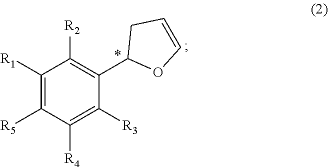
(ii) each * independently represents a chiral center; and
(iii) each of R¹ to R⁵ are independently selected from hydrogen, halo, cyano, unsubstituted C₁-C₆alkyl, substituted C₁-C₆alkyl, unsubstituted C₁-C₆alkoxy, and substituted C₁-C₆alkoxy, wherein at least one of R¹ to R⁵ is halo, wherein at least one halo is selected from Cl and F.

2-12. (canceled)

13. The process of claim 1, wherein each of R¹, R², R³, and R⁴ is hydrogen, and wherein R⁵ is Cl or F.

14-16. (canceled)

17. The process of claim 1, further comprising preparing compound (2), the process comprising forming a reaction mixture comprising compound (1), 2,3-dihydrofuran, a transition metal catalyst, a ligand, a solvent, a base, and reacting the reaction mixture to form a reaction product mixture comprising compound (2), wherein:
(i) compound (1) is of the structure 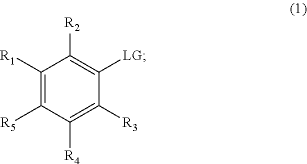
(ii) LG is a leaving group;
(iii) each of R¹ to R⁵ is independently selected from hydrogen, halo, cyano, unsubstituted C₁-C₆alkyl, substituted C₁-C₆alkyl, unsubstituted C₁-C₆alkoxy, and substituted C₁-C₆alkoxy, wherein at least one of R¹ to R⁵ is halo, wherein at least one halo is selected from Cl and F.

18-35. (canceled)

36. The process of claim 1, wherein: compound (2) is of the structure (2a) 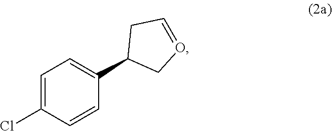
and compound (3) is of the structure (3a)

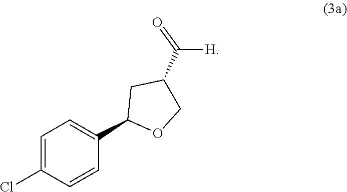

37. The process of claim 17, wherein compound 1 is of the structure (1a) 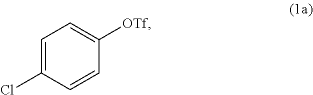 [SMILES: O=S(=O)(Oc1ccc(Cl)cc1)C(F)(F)F]
wherein OTf is trifluoromethanesulfonate.

38. A process for preparing compound (4) 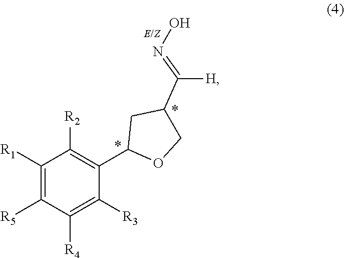
the process comprising forming a reaction mixture comprising a hydroxyl amine solution, a solvent, and compound (3), and reacting the reaction mixture to form a reaction product mixture comprising compound (4),
wherein:
(i) compound (3) is of the structure 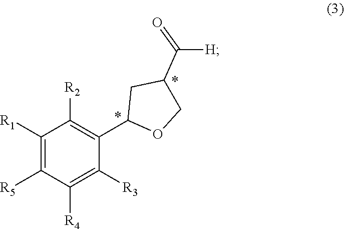
(ii) each * independently represents a chiral center;
(iii) E/Z denotes E/Z isomers; and
(iv) each of R¹ to R⁵ is independently selected from hydrogen, halo, cyano, unsubstituted C₁-C₆alkyl, substituted C₁-C₆alkyl, unsubstituted C₁-C₆alkoxy, and substituted C₁-C₆alkoxy, wherein at least one of R¹ to R⁵ is halo, wherein at least one halo is selected from Cl and F.

39-47. (canceled)

48. The process of claim 38, wherein:
compound (3) is of the structure (3a) 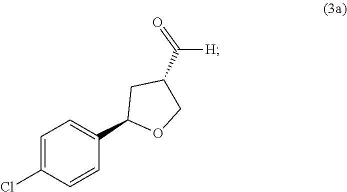
and
compound (4) is of the structure (4a) 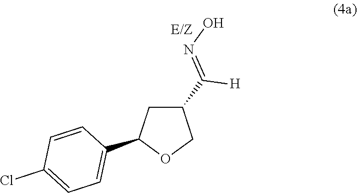 [SMILES: O/N=C/[C@@H]1CO[C@@H](c2ccc(Cl)cc2)C1]
wherein E/Z denotes E/Z isomers.

49. The process of claim 38, further comprising preparing compound (5), the process comprising forming a reaction mixture comprising compound (4), a reagent, and a solvent, and reacting the reaction mixture to form a reaction product mixture comprising compound (5),
wherein:
(i) compound (5) is of the structure 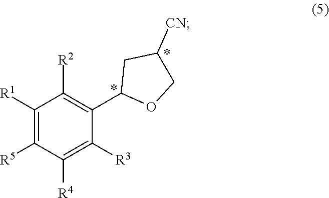
(ii) the reagent is a dehydrating reagent;
(iii) each * independently represents a chiral center;
(iv) each of R¹ to R⁵ are independently selected from hydrogen, halogen, cyano, alkyl, substituted alkyl, alkoxy, and substituted alkoxy, wherein at least one of R¹ to R⁵ is halo; and
(v) at least one of R¹ to R⁵ is selected from Cl and F.

50-56. (canceled)

57. The process of claim 49, wherein:
compound (5) is of the structure (5a)

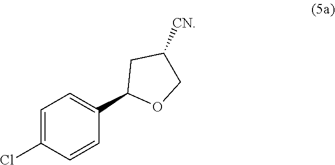 [SMILES: N#C[C@@H]1CO[C@@H](c2ccc(Cl)cc2)C1]

58. The process of claim 49, further comprising preparing compound (6), the process comprising forming a reaction mixture comprising compound (5), a hydroxylamine, and a polar solvent, and reacting the reaction mixture to form a reaction product mixture comprising compound (6),
wherein:
(i) compound (6) is of the structure 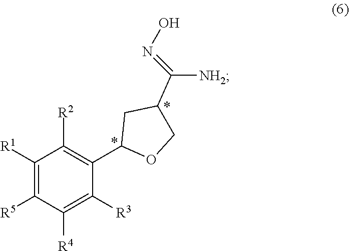
(ii) each * independently represents a chiral center;
(iii) each of R¹ to R⁵ is independently selected from hydrogen, halogen, cyano, alkyl, substituted alkyl, alkoxy, and substituted alkoxy, wherein at least one of R¹ to R⁵ is halo; and
(iv) at least one of R¹ to R⁵ is selected from Cl and F.

59-63. (canceled)

64. The process of claim 58, wherein:
compound (6) is of the structure (6a)

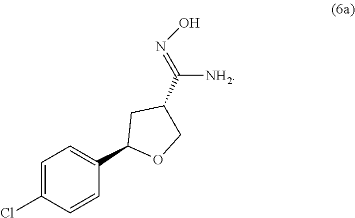 [SMILES: N/C(=N\O)[C@@H]1CO[C@@H](c2ccc(Cl)cc2)C1]

65. The process of claim 38, further comprising preparing compound (7), the process comprising forming a reaction mixture comprising compound (4), an oxidant, an acid, and a solvent, and reacting the reaction mixture to form a reaction product mixture comprising compound (7),
wherein:
(i) compound (7) is of the structure 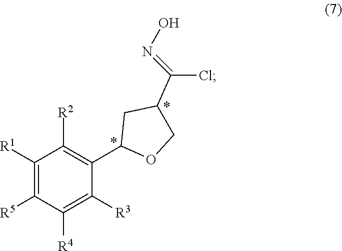
(ii) each * independently represents a chiral center;
(iii) each of R¹ to R⁵ is independently selected from hydrogen, halogen, cyano, alkyl, substituted alkyl, alkoxy, and substituted alkoxy, wherein at least one of R¹ to R⁵ is halo; and
(iv) at least one of R¹ to R⁵ is selected from Cl and F.

66-72. (canceled)

73. The process of claim 65, wherein compound (7) is of the structure compound (7a):

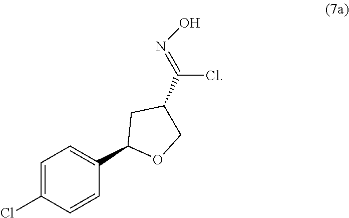 [SMILES: O/N=C(\Cl)[C@@H]1CO[C@@H](c2ccc(Cl)cc2)C1]

74. The process of claim 65, further comprising preparing compound (6), the process comprising forming a reaction mixture comprising compound (7), ammonia, and a polar solvent, and reacting the reaction mixture to form a reaction product mixture comprising compound (6),
wherein:
(i) compound (6) is of the structure 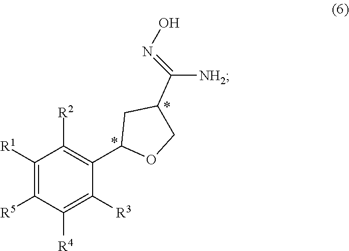
(ii) each * independently represents a chiral center;
(iii) each of R¹, R², R³, and R⁴ are independently selected from hydrogen, halogen, cyano, alkyl, substituted alkyl, alkoxy, and substituted alkoxy, wherein at least one of R¹ to R⁵ is halo; and
(iv) at least one of R¹ to R⁵ is selected from Cl and F.

75-82. (canceled)

83. The process of claim 38, wherein compound (3) is prepared by the process comprising forming a reaction mixture comprising CO, H₂ a rhodium catalyst, a ligand, a solvent and compound (2) and reacting the reaction mixture,
wherein:
(i) compound (2) is of the structure 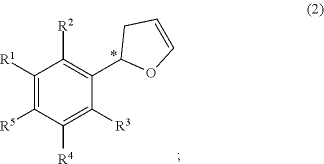
(ii) each * independently represents a chiral center; and
(iii) each of R¹ to R⁵ are independently selected from hydrogen, halo, cyano, unsubstituted C₁-C₆alkyl, substituted C₁-C₆alkyl, unsubstituted C₁-C₆alkoxy, and substituted C₁-C₆alkoxy, wherein at least one of R¹ to R⁵ is halo, wherein at least one halo is selected from Cl and F.

84. A compound of the following structure, or a salt thereof: 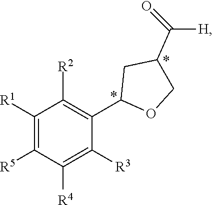
wherein:
(i) each * independently represents a chiral center;
(ii) each of R¹ to R⁵ is independently selected from hydrogen, halogen, cyano, alkyl, substituted alkyl, alkoxy, and substituted alkoxy, wherein at least one of R¹ to R⁵ is halo; and
(iii) at least one of R¹ to R⁵ is selected from Cl and F.

85. The compound of claim 84 of the following structure, or a salt thereof:

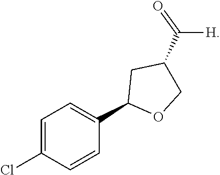

86. A compound of the following structure, or a salt thereof: 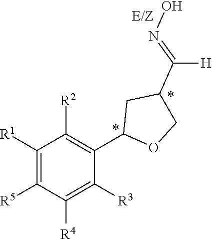
wherein:
(i) each * independently represents a chiral center,
(ii) E/Z denotes E/Z isomers,
(iii) each of R¹ to R⁵ is independently selected from hydrogen, halogen, cyano, alkyl, substituted alkyl, alkoxy, and substituted alkoxy, wherein at least one of R¹ to R⁵ is halo; and
(iv) at least one of R¹ to R⁵ is selected from Cl and F.

87. The compound of claim 86 of the following structure, or a salt thereof: 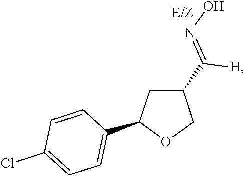
wherein E/Z denotes E/Z isomers.

88. A compound of the following structure, or a salt thereof: 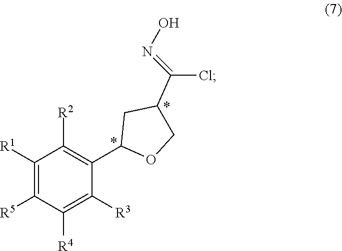
wherein:
(i) each * independently represents a chiral center,
(ii) each of R¹ to R⁵ is independently selected from hydrogen, halogen, cyano, alkyl, substituted alkyl, alkoxy, and substituted alkoxy, wherein at least one of R¹ to R⁵ is halo; and
(iii) at least one of R¹ to R⁵ is selected from Cl and F.

89. The compound of claim 88 of the following structure, or a salt thereof: 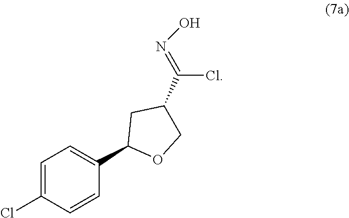 [SMILES: O/N=C(\Cl)[C@@H]1CO[C@@H](c2ccc(Cl)cc2)C1]
